SEQUENCE LISTING (18 sequences)

SEQ ID NO: 1 - protein, 405 aa, Helicobacter pylori
  source 1..405: note = ACJ07781.1
MIGVYIISLKESQRRLDTEKLVSESNEKFKGRCVFQIFDAISPKHEDFEKFVQELYDAQSMLKSDWFHSDYCYQELLPREFGCYLGHYFLWKECVKTNQPVVILEDDVALESNFMQALEDCLKSPFDFVRLYGHYWGGHKTNLCALPIYTEAEVPIENHEVTPPPPNPARDTQQDFIIETQQDPKEPSDPCKIAPQKISFNQVVFKKIKRKLNRFIGSILARTEVYKNVVAKYDDLTKKYDDLTKKYDELTGKYESLLAKETNIKETFWERRADNEKEALFLEHFYLTSVYVATTAGYYLTPKGAKTFIEATERFKIIEPVDMFMNNPTYHDVANFTYLPCPVSLNKHAFNSTIQNAKKPDISLKSPKKSYFDNLFYDQLNTKKCLRAFHKYSKQYAPLKTPKEI

SEQ ID NO: 2 - protein, 265 aa, Escherichia coli
  source 1..265: note = WbgO_YP_003500090
MIIDEAESAESTHPVVSVILPVNKKNPFLDEAINSILSQTFSSFEIIIVANCCTDDFYNELKHKVNDKIKLIRTNIAYLPYSLNKAIDLSNGEFIARMDSDDISHPDRFTKQVDFLKNNPYVDVVGTNAIFIDDKGREINKTKLPEENLDIVKNLPYKCCIVHPSVMFRKKVIASIGGYMFSNYSEDYELWNRLSLAKIKFQNLPEYLFYYRLHEGQSTAKKNLYMVMVNDLVIKMKCFFLTGNINYLFGGIRTIASFIYCKYIK

SEQ ID NO: 3 - protein, 275 aa, Escherichia coli
  source 1..275: note = WbbD_YP_006144407
MSDDTPKFSVLMAIYIKDSPLFLSEALQSIYKNTVAPDEVIIIRDGKVTSELNSVIDSWRRYLNIKDFTLEKNMGLGAALNFGLNQCMHDLVIRADSDDINRTNRFECILDFMTKNGDVHILSSWVEEFEFNPGDKGIIKKVPSRNSILKYSKNRSPFNHPAVAFKKCEIMRVGGYGNEYLYEDYALWLKSLANGCNGDNIQQVLVDMRFSKETAKRRGGIKYAISEIKAQYHFYRANYISYQDFIINIITRIFVRLLPTSFRGYIYKKVIRRFL

SEQ ID NO: 4 - DNA, 6657 bp, Escherichia coli
  source 1..6657: mol_type = genomic DNA; note = E680_thyA__2.8RBS_lacZ
tcacaggttgaatcctgtcacgctatagctggcattcaccacggtttgcggttcagacttactggcagcacgcatattaaccgtcaacaccggcgagaagccgctgacatcctgacgtacgacctgaaaagtgtcgataatgatggcatccggattagtgactttatcccagcccttaccttcacaggatgtcgcaccgcgtagcgtttccagcacatgctccttcagacgaaatccaatctggtcggactcttttaccggttcgcgatcccagataccgttactgttcgcatcccactgcacaatgacacagtcaccctgtccgacaatttccagcccttcgccggtacagatgccatgacaataacccgccctctggagatgcttcgcgacggtaaatacccgcagccagatttcatcttccagcgccagcttacgggtgctcgttaaactttcacgctgtaacgcaggcagaaagcgtgccgcccccagcaacaatacgctactgatcgccatagcaatcaacacttccagcagagaaaaaccttgctcttttacaggcatccttctgtttctccttgctgacaaagccggagtcttccccacggcgaaaccaccagccaccactcgcccgttgagtttttgaagcgaatatgcccggcccatgcggtattgcgcaggccaaagaaagcaagcgaaggtgtcaggtcgctcatttcgacttcgggccagcgtggcacaaagaccaatggtgaactgccatgacaggtattggccccagcagcggaactcacaaggcaccataacgtcccctccctgataacgctgatactgtggtcgcggttatgccagttggcatcttcacgtaaatagagcaaatagtcccgcgcctggctggcggtttgccatagccgttgcgactgctgccagtattgccagccatagagtccacttgcgcttagcatgaccaaaatcagcatcgcgaccagcgtttcaatcagcgtataaccacgttgtgttttcatgccggcagtatggagcgaggagaaaaaaagacgagggccagtttctatttcttcggcgcatcttccggactatttacgccgttgcaggacgttgcaaaatttcgggaaggcgtctcgaagaatttaacggagggtaaaaaaaccgacgcacactggcgtcggctctggcaggatgtttcgtaattagatagccaccggcgctttattaaacctactatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgttgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataagcggccgctttatgtaggctggagctgcttcgaagttcctatactttctagagaataggaacttcggaataggaacttcaagatccccttattagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatcctgaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgagcttcaaaagcgctctgaagttcctatactttctagagaataggaacttcgaactgcaggtcgacggatccccggaatcatggttcctcaggaaacgtgttgctgtgggctgcgacgatatgcccagaccatcatgatcacacccgcgacaatcatcgggatggaaagaatttgccccatgctgatgtactgcacccaggcaccggtaaactgcgcgtcgggctggcggaaaaactcaacaatgatgcgaaacgcgccgtaaccaatcaggaacaaacctgagacagctcccattgggcgtggtttacgaatatacaggttgaggataataaacagcaccacaccttccagcagcagctcgtaaagctgtgatgggtggcgcggcagcacaccgtaagtgtcgaaaatggattgccactgcgggttggtttgcagcagcaaaatatcttctgtacgggagccagggaacagcatggcaaacgggaagttcgggtcaacgcggccccacaattcaccgttaataaagttgcccagacgcccggcaccaagaccaaacggaatgagtggtgcgataaaatcagagacctggaagaaggaacgtttagtacggcgggcgaagataatcatcaccacgataacgccaatcaggccgccgtggaaagacatgccgccgtcccagacacggaacagatacagcggatcggccataaactgcgggaaattgtagaacagaacataaccaatacgtcccccgaggaagacgccgaggaagcccgcatagagtaagttttcaacttcatttttggtccagccgctgcccggacgattcgcccgtcgtgttgccagccacattgcaaaaatgaaacccaccagatacatcaggccgtaccagtgaagcgccacgggtcctattgagaaaatgaccggatcaaactccggaaaatgcagatagctactggtcatctgtcaccacaagttcttgttatttcgctgaaagagaacagcgattgaaatgcgcgccgcaggtttcaggcgctccaaaggtgcgaataatagcacaaggggacctggctggttgccggataccgttaaaagatatgtata

SEQ ID NO: 5 - DNA, 5409 bp, unidentified
  source 1..5409: mol_type = other DNA; note = synthetic plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgcctcctcaacctgtatattcgtaaaccacgcccaatgggagctgtctcaggtttgttcctgattggttacggcgcgtttcgcatcattgttgagtttttccgccagcccgacgcgcagtttaccggtgcctgggtgcagtacatcagcatggggcaaattctttccatcccgatgattgtcgcgggtgtgatcatgatggtctgggcatatcgtcgcagcccacagcaacacgtttcctgaggaaccatgaaacagtatttagaactgatgcaaaaagtgctcgacgaaggcacacagaaaaacgaccgtaccggaaccggaacgctttccatttttggtcatcagatgcgttttaacctgcaagatggattcccgctggtgacaactaaacgttgccacctgcgttccatcatccatgaactgctgtggtttctgcagggcgacactaacattgcttatctacacgaaaacaatgtcaccatctgggacgaatgggccgatgaaaacggcgacctcgggccagtgtatggtaaacagtggcgcgcctggccaacgccagatggtcgtcatattgaccagatcactacggtactgaaccagctgaaaaacgacccggattcgcgccgcattattgtttcagcgtggaacgtaggcgaactggataaaatggcgctggcaccgtgccatgcattcttccagttctatgtggcagacggcaaactctcttgccagctttatcagcgctcctgtgacgtcttcctcggcctgccgttcaacattgccagctacgcgttattggtgcatatgatggcgcagcagtgcgatctggaagtgggtgattttgtctggaccggtggcgacacgcatctgtacagcaaccatatggatcaaactcatctgcaattaagccgcgaaccgcgtccgctgccgaagttgattatcaaacgtaaacccgaatccatcttcgactaccgtttcgaagactttgagattgaaggctacgatccgcatccgggcattaaagcgccggtggctatctaattacgaaacatcctgccagagccgacgccagtgtgcgtcggtttttttaccctccgttaaattcttcgagacgccttcccgaaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttactgctcacaagaaaaaaggcacgtcatctgacgtgccttttttatttgtactaccctgtacgattactgcaggtcgactctagatgcatgctcgagtcaacggtttttcagcaatcggtgcaaaatgccgaagtattgcctcaaggtaaacagccgccgcatcctgccgtctgccgcaaaatccagccacgcgccggcgggcagcgtgtccgtccgtttgaagcattggtacaaaaaccggcgggcgcgttcaaaatcttcttccggcaaatgtttctccagcaattcatacgctactgcttttatttggcggtattcaaggctgtcgaaccgggttttaaaacccatagactgcaaaaaatcgtttctggcggttttttggatgccttgcgcgatttcgtgttggcggatgctgtatttggatgaaacctgattggcgtgaaggcggtatttgaccaaggcttcgggataataagccagcctgcccaatttgctgacatcgtaccaaaattggtaatcttccgcccaatcccgctcggtgttgtaacgcaaaccgccgtcaatgacgctgcgcctcataatcatcgtgttgttgtgtatggggttgccgaaagggaaaaagtcggcaatgtcttcgtgtcgggtcggttttttccaaattttgccgtgttcgtggtgccgcgccagccggttgccgtccttttcttccgacaaaacttccagccacgcacccatcgcgatgatgctgcggtctttttccatctcacccacgattttctcaatccagtcgggggcggcaatatcgtctgcatcggtgcgcgcaatatattccccccccccccccgactttgccaattcatccagcccgatgtttaaagagggaatcagaccggaattgcgcggctgcgcgaggatgcggatgcggccgtcctgttcttggaaacgctgggcaatggcaagcgtaccgtccgtcgagccgtcatcgacaatcaaaatatccaagttgcgccaagtttgattcacgacggcggctaatgattgggcgaaatatttttctacgttgtaggcgcaaatcaatacgctgactaaaggctgcaatttattctcccgataggcacgatgccgtctgaaggcttcagacggcatatgtatatctccttcttgaattctaacaattgattgaatgtatgcaaataaatgcatacaccataggtgtggtttaatttgatgccctttttcagggctggaatgtgtaagagcggggttatttatgctgttgtttttttgttactcgggaagggctttacctcttccgcataaacgcttccatcagcgtttatagttaaaaaaatctttcggaactggttttgcgcttaccccaaccaacaggggatttgctgctttccattgagcctgtttctctgcgcgacgttcgcggcggcgtgtttgtgcatccatctggattctcctgtcagttagctttggtggtgtgtggcagttgtagtcctgaacgaaaaccccccgcgattggcacattggcagctaatccggaatcgcacttacggccaatgcttcgtttcgtatcacacaccccaaagccttctgctttgaatgctgcccttcttcagggcttaatttttaagagcgtcaccttcatggtggtcagtgcgtcctgctgatgtgctcagtatcaccgccagtggtatttatgtcaacaccgccagagataatttatcaccgcagatggttatctgtatgttttttatatgaatttattttttgcaggggggcattgtttggtaggtgagagatcaattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 6 - DNA, 6233 bp, unidentified
  source 1..6233: mol_type = other DNA; note = synthetic plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgcctcctcaacctgtatattcgtaaaccacgcccaatgggagctgtctcaggtttgttcctgattggttacggcgcgtttcgcatcattgttgagtttttccgccagcccgacgcgcagtttaccggtgcctgggtgcagtacatcagcatggggcaaattctttccatcccgatgattgtcgcgggtgtgatcatgatggtctgggcatatcgtcgcagcccacagcaacacgtttcctgaggaaccatgaaacagtatttagaactgatgcaaaaagtgctcgacgaaggcacacagaaaaacgaccgtaccggaaccggaacgctttccatttttggtcatcagatgcgttttaacctgcaagatggattcccgctggtgacaactaaacgttgccacctgcgttccatcatccatgaactgctgtggtttctgcagggcgacactaacattgcttatctacacgaaaacaatgtcaccatctgggacgaatgggccgatgaaaacggcgacctcgggccagtgtatggtaaacagtggcgcgcctggccaacgccagatggtcgtcatattgaccagatcactacggtactgaaccagctgaaaaacgacccggattcgcgccgcattattgtttcagcgtggaacgtaggcgaactggataaaatggcgctggcaccgtgccatgcattcttccagttctatgtggcagacggcaaactctcttgccagctttatcagcgctcctgtgacgtcttcctcggcctgccgttcaacattgccagctacgcgttattggtgcatatgatggcgcagcagtgcgatctggaagtgggtgattttgtctggaccggtggcgacacgcatctgtacagcaaccatatggatcaaactcatctgcaattaagccgcgaaccgcgtccgctgccgaagttgattatcaaacgtaaacccgaatccatcttcgactaccgtttcgaagactttgagattgaaggctacgatccgcatccgggcattaaagcgccggtggctatctaattacgaaacatcctgccagagccgacgccagtgtgcgtcggtttttttaccctccgttaaattcttcgagacgccttcccgaaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttactgctcacaagaaaaaaggcacgtcatctgacgtgccttttttatttgtactaccctgtacgattactgcaggtcgactctagatgcatgctcgagttattatttaatatatttacaatagatgaaggacgcaatcgtacggataccgccgaacaggtagttaatgttaccggtcaggaagaagcacttcattttgataaccaggtcgttaaccatcaccatgtacaggtttttttttgcggtagactgaccttcgtgcaggcggtagtagaacaggtattccggcaggttttggaacttgatttttgccaggctcagacggttccacagctcgtaatcttcggagtagttagaaaacatataaccaccgatgctcgcgatgacttttttacgaaacattacgctcgggtgaacaatacaacacttatacggcaggtttttaacgatgtccaggttctcttccggcagtttggtcttgttgatttcacgacctttgtcgtcaataaagattgcgttggtacccacaacatctacgtacggattgttcttcaggaagtcaacctgtttagtaaaacggtccgggtgagagatgtcgtcagagtccatacgggcaataaattcgccgttgctcaggtcgatcgctttgttcagggagtacggcaggtaagcgatgttagtgcggatcagtttgattttgtcgttaactttgtgtttcagttcgttatagaagtcgtcagtgcagcagttcgcaacgatgatgatttcgaagctgctgaaggtctgagacaggatgctgttgatcgcttcgtccagaaaagggtttttcttgttaacaggcaggataacgctcacaaccgggtgggtagattccgcggattccgcttcatcgatgatcatatgtatatctccttcttctcgagtcaacggtttttcagcaatcggtgcaaaatgccgaagtattgcctcaaggtaaacagccgccgcatcctgccgtctgccgcaaaatccagccacgcgccggcgggcagcgtgtccgtccgtttgaagcattggtacaaaaaccggcgggcgcgttcaaaatcttcttccggcaaatgtttctccagcaattcatacgctactgcttttatttggcggtattcaaggctgtcgaaccgggttttaaaacccatagactgcaaaaaatcgtttctggcggttttttggatgccttgcgcgatttcgtgttggcggatgctgtatttggatgaaacctgattggcgtgaaggcggtatttgaccaaggcttcgggataataagccagcctgcccaatttgctgacatcgtaccaaaattggtaatcttccgcccaatcccgctcggtgttgtaacgcaaaccgccgtcaatgacgctgcgcctcataatcatcgtgttgttgtgtatggggttgccgaaagggaaaaagtcggcaatgtcttcgtgtcgggtcggttttttccaaattttgccgtgttcgtggtgccgcgccagccggttgccgtccttttcttccgacaaaacttccagccacgcacccatcgcgatgatgctgcggtctttttccatctcacccacgattttctcaatccagtcgggggcggcaatatcgtctgcatcggtgcgcgcaatatattccccccccccccccgactttgccaattcatccagcccgatgtttaaagagggaatcagaccggaattgcgcggctgcgcgaggatgcggatgcggccgtcctgttcttggaaacgctgggcaatggcaagcgtaccgtccgtcgagccgtcatcgacaatcaaaatatccaagttgcgccaagtttgattcacgacggcggctaatgattgggcgaaatatttttctacgttgtaggcgcaaatcaatacgctgactaaaggctgcaatttattctcccgataggcacgatgccgtctgaaggcttcagacggcatatgtatatctccttcttgaattctaacaattgattgaatgtatgcaaataaatgcatacaccataggtgtggtttaatttgatgccctttttcagggctggaatgtgtaagagcggggttatttatgctgttgtttttttgttactcgggaagggctttacctcttccgcataaacgcttccatcagcgtttatagttaaaaaaatctttcggaactggttttgcgcttaccccaaccaacaggggatttgctgctttccattgagcctgtttctctgcgcgacgttcgcggcggcgtgtttgtgcatccatctggattctcctgtcagttagctttggtggtgtgtggcagttgtagtcctgaacgaaaaccccccgcgattggcacattggcagctaatccggaatcgcacttacggccaatgcttcgtttcgtatcacacaccccaaagccttctgctttgaatgctgcccttcttcagggcttaatttttaagagcgtcaccttcatggtggtcagtgcgtcctgctgatgtgctcagtatcaccgccagtggtatttatgtcaacaccgccagagataatttatcaccgcagatggttatctgtatgttttttatatgaatttattttttgcaggggggcattgtttggtaggtgagagatcaattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 7 - DNA, 6263 bp, unidentified
  source 1..6263: mol_type = other DNA; note = synthetic plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgcctcctcaacctgtatattcgtaaaccacgcccaatgggagctgtctcaggtttgttcctgattggttacggcgcgtttcgcatcattgttgagtttttccgccagcccgacgcgcagtttaccggtgcctgggtgcagtacatcagcatggggcaaattctttccatcccgatgattgtcgcgggtgtgatcatgatggtctgggcatatcgtcgcagcccacagcaacacgtttcctgaggaaccatgaaacagtatttagaactgatgcaaaaagtgctcgacgaaggcacacagaaaaacgaccgtaccggaaccggaacgctttccatttttggtcatcagatgcgttttaacctgcaagatggattcccgctggtgacaactaaacgttgccacctgcgttccatcatccatgaactgctgtggtttctgcagggcgacactaacattgcttatctacacgaaaacaatgtcaccatctgggacgaatgggccgatgaaaacggcgacctcgggccagtgtatggtaaacagtggcgcgcctggccaacgccagatggtcgtcatattgaccagatcactacggtactgaaccagctgaaaaacgacccggattcgcgccgcattattgtttcagcgtggaacgtaggcgaactggataaaatggcgctggcaccgtgccatgcattcttccagttctatgtggcagacggcaaactctcttgccagctttatcagcgctcctgtgacgtcttcctcggcctgccgttcaacattgccagctacgcgttattggtgcatatgatggcgcagcagtgcgatctggaagtgggtgattttgtctggaccggtggcgacacgcatctgtacagcaaccatatggatcaaactcatctgcaattaagccgcgaaccgcgtccgctgccgaagttgattatcaaacgtaaacccgaatccatcttcgactaccgtttcgaagactttgagattgaaggctacgatccgcatccgggcattaaagcgccggtggctatctaattacgaaacatcctgccagagccgacgccagtgtgcgtcggtttttttaccctccgttaaattcttcgagacgccttcccgaaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttactgctcacaagaaaaaaggcacgtcatctgacgtgccttttttatttgtactaccctgtacgattactgcaggtcgactctagatgcatgctcgagttactataaaaatctcctgataacttttttatatatatagccacgaaaactagtgggaagaagtctaacaaatatccttgtgataatatttattataaagtcttgatatgatatataatttgcacgataaaaatgatactgagctttaatttctgaaatggcatattttattccacctcgtctttttgctgtttcctttgaaaatctcatatcaactaaaacttgttgaatattatcaccattacatccattagctaaagatttcaaccaaagggcataatcttcatataagtactcatttccataaccgccgacgcgcattatttcacactttttaaatgcaactgcagggtgattaaaaggagatctgttttttgaatatttaagtatagaattccgacttggtactttttttattatgcccttatctcctggattgaattcgaactcttcaacccaagagctaagaatatgaacatcaccattcttagtcataaaatcaagtatacattcgaatcgatttgttctatttatatcatcagaatcagcacgtattactaaatcatgcatacattgattcaacccaaaatttaacgctgcccccaaccccatatttttttcaagtgtgaaatcttttatatttaaatatcttctccaactatcaataacagaattgagttcagatgtgaccttaccatcacgaataataataacttcatctggggcaaccgtatttttataaattgattgtaaagcctcagagagaaataggggagaatccttgatgtatatagccatcaaaacagaaaactttggagtgtcatctgacatatgtatatctccttcttctcgagtcaacggtttttcagcaatcggtgcaaaatgccgaagtattgcctcaaggtaaacagccgccgcatcctgccgtctgccgcaaaatccagccacgcgccggcgggcagcgtgtccgtccgtttgaagcattggtacaaaaaccggcgggcgcgttcaaaatcttcttccggcaaatgtttctccagcaattcatacgctactgcttttatttggcggtattcaaggctgtcgaaccgggttttaaaacccatagactgcaaaaaatcgtttctggcggttttttggatgccttgcgcgatttcgtgttggcggatgctgtatttggatgaaacctgattggcgtgaaggcggtatttgaccaaggcttcgggataataagccagcctgcccaatttgctgacatcgtaccaaaattggtaatcttccgcccaatcccgctcggtgttgtaacgcaaaccgccgtcaatgacgctgcgcctcataatcatcgtgttgttgtgtatggggttgccgaaagggaaaaagtcggcaatgtcttcgtgtcgggtcggttttttccaaattttgccgtgttcgtggtgccgcgccagccggttgccgtccttttcttccgacaaaacttccagccacgcacccatcgcgatgatgctgcggtctttttccatctcacccacgattttctcaatccagtcgggggcggcaatatcgtctgcatcggtgcgcgcaatatattccccccccccccccgactttgccaattcatccagcccgatgtttaaagagggaatcagaccggaattgcgcggctgcgcgaggatgcggatgcggccgtcctgttcttggaaacgctgggcaatggcaagcgtaccgtccgtcgagccgtcatcgacaatcaaaatatccaagttgcgccaagtttgattcacgacggcggctaatgattgggcgaaatatttttctacgttgtaggcgcaaatcaatacgctgactaaaggctgcaatttattctcccgataggcacgatgccgtctgaaggcttcagacggcatatgtatatctccttcttgaattctaacaattgattgaatgtatgcaaataaatgcatacaccataggtgtggtttaatttgatgccctttttcagggctggaatgtgtaagagcggggttatttatgctgttgtttttttgttactcgggaagggctttacctcttccgcataaacgcttccatcagcgtttatagttaaaaaaatctttcggaactggttttgcgcttaccccaaccaacaggggatttgctgctttccattgagcctgtttctctgcgcgacgttcgcggcggcgtgtttgtgcatccatctggattctcctgtcagttagctttggtggtgtgtggcagttgtagtcctgaacgaaaaccccccgcgattggcacattggcagctaatccggaatcgcacttacggccaatgcttcgtttcgtatcacacaccccaaagccttctgctttgaatgctgcccttcttcagggcttaatttttaagagcgtcaccttcatggtggtcagtgcgtcctgctgatgtgctcagtatcaccgccagtggtatttatgtcaacaccgccagagataatttatcaccgcagatggttatctgtatgttttttatatgaatttattttttgcaggggggcattgtttggtaggtgagagatcaattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 8 - DNA, 6653 bp, unidentified
  source 1..6653: mol_type = other DNA; note = synthetic plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgcctcctcaacctgtatattcgtaaaccacgcccaatgggagctgtctcaggtttgttcctgattggttacggcgcgtttcgcatcattgttgagtttttccgccagcccgacgcgcagtttaccggtgcctgggtgcagtacatcagcatggggcaaattctttccatcccgatgattgtcgcgggtgtgatcatgatggtctgggcatatcgtcgcagcccacagcaacacgtttcctgaggaaccatgaaacagtatttagaactgatgcaaaaagtgctcgacgaaggcacacagaaaaacgaccgtaccggaaccggaacgctttccatttttggtcatcagatgcgttttaacctgcaagatggattcccgctggtgacaactaaacgttgccacctgcgttccatcatccatgaactgctgtggtttctgcagggcgacactaacattgcttatctacacgaaaacaatgtcaccatctgggacgaatgggccgatgaaaacggcgacctcgggccagtgtatggtaaacagtggcgcgcctggccaacgccagatggtcgtcatattgaccagatcactacggtactgaaccagctgaaaaacgacccggattcgcgccgcattattgtttcagcgtggaacgtaggcgaactggataaaatggcgctggcaccgtgccatgcattcttccagttctatgtggcagacggcaaactctcttgccagctttatcagcgctcctgtgacgtcttcctcggcctgccgttcaacattgccagctacgcgttattggtgcatatgatggcgcagcagtgcgatctggaagtgggtgattttgtctggaccggtggcgacacgcatctgtacagcaaccatatggatcaaactcatctgcaattaagccgcgaaccgcgtccgctgccgaagttgattatcaaacgtaaacccgaatccatcttcgactaccgtttcgaagactttgagattgaaggctacgatccgcatccgggcattaaagcgccggtggctatctaattacgaaacatcctgccagagccgacgccagtgtgcgtcggtttttttaccctccgttaaattcttcgagacgccttcccgaaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttactgctcacaagaaaaaaggcacgtcatctgacgtgccttttttatttgtactaccctgtacgattactgcaggtcgactctagatgcatgctcgagttattagatttccttcggcgtcttcagcggggcatactgcttagaatacttatggaatgcacgcagacatttcttggtattcagctggtcgtagaataagttgtcgaagtagctcttcttcgggcttttcaggctgatatctggtttcttagcgttctggatcgtagagttaaacgcgtgtttattcaggcttaccggacacggtaggtaagtgaagttcgcaacgtcgtgataggtcggattattcataaacatgtcaacaggttcgataatcttgaatctttcggtcgcctcgatgaaagtctttgcacctttcggagtcaaataataaccagcggtcgttgcaacgtagacagaagtcaaatagaagtgttccaggaacaaagcctccttctcattgtccgctctgcgctcccagaaggtttccttaatattggtttccttcgctagcagactctcatacttacccgttaactcgtcatacttcttcgttaaatcgtcatacttcttagttaaatcgtcatacttcgcaaccacgttcttgtagacttcggtacgcgccaggatggagccgataaaacggttcagtttgcgtttgatttttttgaaaactacctggttgaagctaattttctgcggcgcgatcttacacgggtcgctcggttccttcggatcctgctgagtctcgatgatgaagtcctgctgggtgtccctggccgggttcggcggcggtggagttacctcgtgattctcgatcggtacctccgcttcagtgtagatcggcaacgcacataggttcgtcttgtgaccaccccagtagtggccatacaggcgaacgaagtcgaacggagactttagacagtcctccagagcctgcataaagttgctttccagagcgacgtcgtcctccaggatgacaactggctgattagtctttacacactccttccataggaagtagtgacccaggtaacaaccgaactcacgcggtagcagctcctggtagcagtagtcgctgtgaaaccagtcactcttcagcatggactgggcgtcgtacaattcctggacgaacttctcgaagtcctcatgcttcggagagatcgcatcgaaaatctggaatacacatctacccttgaatttctcgttactctcactgaccaacttctcggtgtctagcctacgctgggactccttcaggctgataatgtatacgccgatcatatgtatatctccttcttctcgagtcaacggtttttcagcaatcggtgcaaaatgccgaagtattgcctcaaggtaaacagccgccgcatcctgccgtctgccgcaaaatccagccacgcgccggcgggcagcgtgtccgtccgtttgaagcattggtacaaaaaccggcgggcgcgttcaaaatcttcttccggcaaatgtttctccagcaattcatacgctactgcttttatttggcggtattcaaggctgtcgaaccgggttttaaaacccatagactgcaaaaaatcgtttctggcggttttttggatgccttgcgcgatttcgtgttggcggatgctgtatttggatgaaacctgattggcgtgaaggcggtatttgaccaaggcttcgggataataagccagcctgcccaatttgctgacatcgtaccaaaattggtaatcttccgcccaatcccgctcggtgttgtaacgcaaaccgccgtcaatgacgctgcgcctcataatcatcgtgttgttgtgtatggggttgccgaaagggaaaaagtcggcaatgtcttcgtgtcgggtcggttttttccaaattttgccgtgttcgtggtgccgcgccagccggttgccgtccttttcttccgacaaaacttccagccacgcacccatcgcgatgatgctgcggtctttttccatctcacccacgattttctcaatccagtcgggggcggcaatatcgtctgcatcggtgcgcgcaatatattccccccccccccccgactttgccaattcatccagcccgatgtttaaagagggaatcagaccggaattgcgcggctgcgcgaggatgcggatgcggccgtcctgttcttggaaacgctgggcaatggcaagcgtaccgtccgtcgagccgtcatcgacaatcaaaatatccaagttgcgccaagtttgattcacgacggcggctaatgattgggcgaaatatttttctacgttgtaggcgcaaatcaatacgctgactaaaggctgcaatttattctcccgataggcacgatgccgtctgaaggcttcagacggcatatgtatatctccttcttgaattctaacaattgattgaatgtatgcaaataaatgcatacaccataggtgtggtttaatttgatgccctttttcagggctggaatgtgtaagagcggggttatttatgctgttgtttttttgttactcgggaagggctttacctcttccgcataaacgcttccatcagcgtttatagttaaaaaaatctttcggaactggttttgcgcttaccccaaccaacaggggatttgctgctttccattgagcctgtttctctgcgcgacgttcgcggcggcgtgtttgtgcatccatctggattctcctgtcagttagctttggtggtgtgtggcagttgtagtcctgaacgaaaaccccccgcgattggcacattggcagctaatccggaatcgcacttacggccaatgcttcgtttcgtatcacacaccccaaagccttctgctttgaatgctgcccttcttcagggcttaatttttaagagcgtcaccttcatggtggtcagtgcgtcctgctgatgtgctcagtatcaccgccagtggtatttatgtcaacaccgccagagataatttatcaccgcagatggttatctgtatgttttttatatgaatttattttttgcaggggggcattgtttggtaggtgagagatcaattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 9 - protein, 427 aa, Helicobacter pylori
  source 1..427: note = WP_033756231.1
MIGVYIISLKESQRRLDTEKLILESNEKFKGRCVFQIFDAISPKHEDFEKFVQELYDAQSMLKSDWFHSDWCRGELLPQEFGCYLSHYLLWKECVKLNQPVVILEDDVALESNFMQALEDCLKSPFDFVKLFGWYWNFHKTNLRTLPLERDAVESVGETPIEDHVKTEAPETPIENHEVTPPPNPARDAQQDFIIETQQEELSEPCKIAPQKISFNQVVFKKIKRKLNHFIGNILARTEVYKKLTGKYDELTGKYDELTGKYDELTGKYDELTGKYESLLAKETNIKETFWERRADSEEEAFFLEHFYLTSVYVASTAGYYITPKGAKTFIEATERFKIIEPVDMFINNPTYHDVATLTYLPLPVSLNKHCKISTIQNLKKSDISLSGPKKSYLDNLLYDQLNTRKCLKAFHKYSKQYAPLKTPKEI

SEQ ID NO: 10 - protein, 350 aa, Helicobacter cetorum
  source 1..350: note = WP_104713491.1
MTQVYIISLKDSKRRLDTEELVSQANIDFEGHCAFHIFDAISPKHKDFEELVREFYEPKSLLKSDWFHSDCCNGGLLPQELGCFLSHYFLWKKCLELNEPIIILEDDVALEPNFIQALKDCLKSPFEFVRFCGDYWGYHHTYLNALPIYDNGITPPPPNEESQPIQGSFLAHMVHRVLYFIIYKIFNRIFHLSLYSIVYRFSRIIKNLQRSHYKKYEKETFFLEHFYLTSVYVGRTAGYYLTPKGAKAFVDATRNFKMIEPVDMFMDNPAYTDIASITYIPCALSLNEHSLNSTIANQKPELLKSYALPKAPKKSYFKNLFYYALNARKRQKAFKKFYEKYAYLKSCKDF

SEQ ID NO: 11 - protein, 246 aa, Helicobacter fennelliae
  source 1..246: note = WP_023949252.1
MFHIFIISLQNSPRRAFMQEQCTHLDRGICQVHFFDAIDFRTNAYPALNSKIKPLWNRIYWGRELSISELGCFGSHYSLWEKCIELNAPIIVLEDDVKLESFFMQGLQEIDQSGFEYVRLMGLFDVKIEPIKTKSAESKLAESTTKTQHFFKTTDQIAGTQGYYLTPNAAKKFIAKLHSFCMPVDDYMDCFFIHKVGNILYKPYLIAPAELESTISGRIKQPFSVFKITRECFRLWRKLRRLLHCL

SEQ ID NO: 12 - protein, 254 aa, Campylobacter jejuni
  source 1..254: note = OEV48919.1
MKVFIINLERSLDRKKHMQKQIQKLFEKNPSLKNKLEFIFFKAIDAKNKEHLEFKDHFSWWGSWILGRELSDGEKACFASHYKLWQECVKLDEPIIILEDDVEFSDEFLNNGIEYIDELLKSKYEYIRLCYLFDKRLYFLSEGGYYLSFEKLAGTQGYVLQVSAAKKFLKCAKNWIYAVDDYMDMFYKHNVLNIVKRPLFLKQANFSSVIVEYGRKFSIKLILYKKIAREIFRFYSNILRLLSIVYIKNRLKLK

SEQ ID NO: 13 - protein, 242 aa, Vibrio cholerae
  source 1..242: note = WP_002023705.1
MKIYVISLKNSLDRRASIEQQMTSHGLKFEFFDAIDGRIDPPHPLFANYDYIKRLWLTSGKMPMRGELGCYASHYLLWQKCVELNAPIVVLEDDVIINENFSQYLSIIKDKTNEYGFLRLEPEVGKCSLFSKESKENYSIAFMDNNWGGTRAYSISPDSARKLILGSQKWSMAVDNYIGCTYIHKMPSYIFSPSMVEHGVEFETTFQNEKRIRVPLYRKPTREIYSVYKKIRIMMFANEYKK

SEQ ID NO: 14 - protein, 249 aa, Gallibacterium anatis
  source 1..249: note = WP_018346553.1
MLPIYVIHIDSATERADSIRQQFDNLKIEFEFFPAINAKKTPNHPLFSHYNAKKHFQRKGRNLSSGELGCYASHYSTWKKCLELNQPIIVLEDDVTILENFKDIYTNAERIIQKYDFVWLHKNHRSDDKVIVESIDAFSIAKFYRDYFCAQGYLITPKAAKQLLTYCEEWIYPVDDQMGRFYENKIENYAIYPACIDHIASMESLIGDDRRGKKKLSFTSKIRREYFNLKDHCRRAWYNFCFKLGAEVD

SEQ ID NO: 15 - protein, 452 aa, Helicobacter pylori
MVECQRIPYLGVHLIQVYIISLKESQRRLDTEKLVLESNEKFKGRCVFQIFDAISPKHQDFEKFVQELYDAQSMLKSDWFHSDYCYQELLPQELGCYLSHYLLWKECVKTDQPIVILEDDVALESNFMQALEDCLKSPFDFVRLYGHYWGGHKTNLCALPIYTEAEETDYIETEAPIENHEVTPPPPNPAQDTQQDLINETQQKEPSEPCKIAPPKISFNQVVFKKIKRKLNHFIGNILARTEVHKKLVAKYDELTGKYDELTGKYDELTGKYDELTGKYDELTGKYDELTGKYDELTGKYESLLAKESNIKETFWERRADSEKEAFFLEHFYLTSVYVSTTAGYYLTPKGAKTFIEATERFKIIEPVDMFINNPTYHDIANFTYVPCPVSLNKHAFNSTIQNAKKPDISLKPPKKSYFDNLFYNQLNTRKCLRAFHKYSKQYAPLKTPKEV

SEQ ID NO: 16 - protein, 481 aa, Helicobacter pylori
MISVYIISLKESQRRLDTEKLVLESNEKFKGRCVFQIFDAISPKHEDFEKLLQELYDSSNLLKSDWFHSDYCYQELLPQEFGCYLSHYLLWKECVKTNQPVVILEDDIALESNFMQALEDCLKSPFDFVRLYGHYWGGHKTNLCALPIYTENENEEVEVPMENHAETEASMEKTPIENHEVTPPPPNPTQDAQQDCIIETQQDPKELSEPCKIAPQKTSFNPVVFRKIKRKLNRFIGNILARTEVYKNLVSKYDELTGKYDELTGKYDELTGKYDELTGKYDELTGKYDELTGKYDELTGKYDELTGKYDELTGKYDELTGKYDELTGKYESLLAKEVNIKETFWESRADSEKEALFLEHFYLTSVYVATTAGYYLTPKGAKTFIEATERFKIIEPVDMFINNPTYHDVANFTYLPCPVSLNKHAFNSTIQNAKKPDISLKPPKKSYFDNLFYHKFNAQKCLKAFHKYSKQYAPLKTPKEV

SEQ ID NO: 17 - protein, 444 aa, Helicobacter pylori
  source 1..444: note = WP_075667830.1
MIQVYIISLKESQRRLDTEKLVLESNEKFKGRCVFQIFDAISPKHQDFEKFVQELYDAQSMLKSDWFHSDYCYQELLPREFGCYLSHYLLWKECVKTNQPVVILEDDVALESNFMQALEDCLKSPFDFVRLYGHYWGGHKTNLCALPIYTEIEETDYTEIEEAEAPIENHEVPPPPPNSTQDTQQDLINETQQNPKEPSNPCKIAPQKVSFNQVVFKKIKRKLNHFIGNILARTEVYKKLVAKYDDLTGKYDELTGKYDELTGKYDELTGKYDELTGKYDELTGKYDELTGKYESLLAKEANIKETFWERRADSEKEAFFLEHFYLTSVYVSTTAGYYITPKGAKTFIEATERFKIIEPVDMFINNPTYHDIANFTYVPCPISLNKHAFNSTIQNAKKPDISLKPPKKSYWDNLFYNQLNTKKCLRAFHKYSKQYDHLKTPKEV

SEQ ID NO: 18 - protein, 334 aa, Helicobacter cetorum
  source 1..334: note = WP_014659558.1
MISVYIISLKDSKRRLDTEKLVLESNEKFRGHCVFHIFDAISPKHEDFEKLVKELYDASSLLQSDWFCSSVGNGLSLPELGCYLSHYFLWEECAKLNQPVIVLEDDVALESNFIQALEDCLKSPFDFVRLYGDYWYFHSTDFNTLFTQTANTEKNFKYYIKSRLKNLFKSIPLSQIIIRIPTKTAELFQRKYFSKREKEALFLEHFYLTSVYVATTAGYYLTPKGAKTFIDATKKFKIIEPVDMFMDNPTYHDVASLTYVPCALSLNGHSFNSTIQSQHQGNKKENKKRYKIVLPTPPRKAYLKRLFSYATNAKKRLKAFQQFYEKYAHLESHT

(19) United States
(12) Patent Application Publication
(10) Pub. No.: US 2024/0084246 A1
(43) Pub. Date: Mar. 14, 2024

(54) ß-1,3-GALACTOSYLTRANSFERASES FOR USE IN THE BIOSYNTHESIS OF OLIGOSACCHARIDES

(71) Applicant: Glycosyn LLC, Waltham, MA (US)

(72) Inventors: Kelly A. Miller, Burlington, MA (US); John M. McCoy, Reading, MA (US)

(21) Appl. No.: 18/456,115

(22) Filed: Aug. 25, 2023

Related U.S. Application Data
(60) Provisional application No. 63/373,468, filed on Aug. 25, 2022.

(51) Int. Cl. C12N 1/20; C12N 9/10; C12P 19/04

(52) U.S. Cl. CPC C12N 1/205; C12N 9/1051; C12P 19/04; C12Y 204/01179

(57) ABSTRACT

Methods and compositions for the production of Type 1 human milk oligosaccharides are described.

### RELATED APPLICATION

This application claims the benefit of U.S. Provisional Application No. 63/373,468 filed on Aug. 25, 2022. The entire teachings of the above application(s) are incorporated herein by reference.

### INCORPORATION BY REFERENCE OF MATERIAL IN XML

This application incorporates by reference the Sequence Listing contained in the following eXtensible Markup Language (XML) file being submitted concurrently herewith:
a) File name: 62271009001_Corrected_Sequence_Listing.xml; created Oct. 10, 2023, 54,985 Bytes in size.

### BACKGROUND

Human milk contains a diverse set of neutral and acidic sugar oligomers collectively known as the “human milk oligosaccharides” (HMOs) (Bode and Jantscher-Krenn, 2012; Chaturvedi et al., 1997; Cheng et al., 2020; Kunz et al., 2000). More than 200 distinct oligosaccharide species have been identified in human milk, and both their particular complement of structural features and their high overall abundance are unique to humans. Although these HMO sugars are not utilized per se by infants for nutrition, they nevertheless serve critical roles in the establishment of a healthy infant gut microbiome, in the prevention of disease, and in immune function (Bode and Jantscher-Krenn, 2012; Cheng et al., 2020; Gnoth et al., 2000; Newburg and Walker, 2007; Ray et al., 2019; Rudloff and Kunz, 2012).

Lacto-N-tetraose (LNT) is one of the major individual human milk oligosaccharide species and contains within its structure the most abundant HMO foundational motif (i.e. Gal(β1-3)GlcNAc), a motif called the “Type 1” glycan core. The related, but distinct, “Type 2” glycan core structure (i.e. Gal(β1-4)GlcNAc) is rarer, and is found in a smaller subset of HMOs. Most of the higher molecular weight oligosaccharides in human milk, i.e., those larger in size than three combined hexose units, are based on LNT, and therefore include the Type 1 core structure. Thus, the ability to synthesize the (Gal(β1-3)GlcNAc) motif is critically important for the production of the broadest selection of HMOs.

### SUMMARY

Prior to the disclosure described herein, the ability to produce certain “Type 1” human milk oligosaccharides inexpensively was problematic. Indeed, their production through chemical synthesis remains limited by stereo-specificity issues, precursor availability, product impurities, and high overall cost. As such, there exists a continuing need for new tools and strategies to inexpensively manufacture large quantities of LNT and its derived Type 1 HMOs.

Accordingly, the disclosure features newly discovered LNT2-accepting β-1,3-galactosyltransferase enzymes, GatA (SEQ ID NO:1), GatB (SEQ ID NO:17), GatC (SEQ ID NO:10), and GatD (SEQ ID NO:18). These enzymes are useful for cost-effective and efficient biosynthesis of oligosaccharides.

In addition to the amino acid sequences described above, the disclosure also encompasses enzymes that are less than 100% identical to the reference sequence of SEQ ID NO: 1, 17, 10, or 18. For example, such an amino acid sequence comprises at least 50% sequence identity to the reference sequence and retain β-1,3-galactosyltransferase activity. In some examples, the sequence is at least 60%, 75%, 80%, 85%, 90%, 95%, and 99% identical to the reference sequence, e.g., SEQ ID NO: 1, 17, 10, or 18 and retain β-1,3-galactosyltransferase activity.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Percent identity is determined using search algorithms such as BLAST and PSI-BLAST (Altschul et al., 1990, J Mol Biol 215:3, 403-410; Altschul et al., 1997, Nucleic Acids Res 25:17, 3389-402). For the PSI-BLAST search, the following exemplary parameters are employed: (1) Expect threshold was 10; (2) Gap cost was Existence:11 and Extension:1; (3) The Matrix employed was BLOSUM62; (4) The filter for low complexity regions was “on”.

The β-1,3-galactosyltransferases of the disclosure include the amino acid sequences of SEQ ID NOs: 1, 17, 10, or 18 as well as fragments and variants thereof that exhibit β-1,3-galactosyltransferase activity.

The disclosure provides methods for producing oligosaccharides that comprise a Type 1 glycan core, i.e. Gal(β1-3)GlcNAc, (e.g., LNT or its derived Type 1 HMOs) or a Type 2 glycan core, i.e. Gal(β1-4)GlcNAc. The methods comprise providing a bacterium that expresses at least one exogenous LNT-accepting β-1,3-galactosyltransferase and culturing the bacterium to inexpensively and efficiently produce oligosaccharides. The methods may further comprise retrieving or purifying the oligosaccharide from the bacterium or from a culture supernatant of the bacterium.

For example, the disclosure includes methods for producing an oligosaccharide in a bacterium comprising expressing an enzyme in a host bacterium, wherein the amino acid sequence of said enzyme comprises at least 85% identity to GatB (SEQ ID NO:17), thereby producing an oligosaccharide comprising a Gal(β1-3)GlcNAc motif The disclosure also encompasses compositions for use in the production of an oligosaccharide, the composition comprising a bacterium expressing at least one β-1,3-galactosyltransferase enzyme, wherein the amino acid sequence of said at least one enzyme comprises at least 80% identity, at least 85%, at least 90%, at least 95%, at least 99%, and up to 100% identity to full length amino acid sequence of SEQ ID NO: 1, 17, 10, or 18. Biosynthetic oligosaccharides produced according to the disclosure are useful as ingredients in nutritional supplements and/or therapeutics.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a diagram of synthetic routes for neutral hMOS.

FIG. 2 is a diagram of synthetic routes for acidic hMOS.

FIG. 3 is a diagram of Type 1 and Type 2 glycan motifs.

FIG. 4 is a diagram of a configuration of genes engineered at the thyA gene locus.

FIG. 5 is a diagram of the first step in the production of lacto-N-tetraose (LNT) in E. coli.

FIG. 6 is a schematic diagram of an exemplary plasmid, pG292, used for production of LNT2.

FIG. 7 is a diagram showing the conversion of LNT2 to LNT by a β(1,3) galactosyltransferase.

FIG. 8 is a schematic diagram of an exemplary plasmid, pG221, for production of LNT.

FIG. 9 is a photograph of a thin layer chromatogram showing production of LNT2 and LNT.

FIG. 10 is a photograph of a thin layer chromatogram showing LNT production: comparison of β-1,3 galactosyltransferases WbgO and WbbD with newly discovered β-1,3 galactosyltransferase GatA

FIG. 11 is a table of pairwise amino acid sequence identity comparisons to GatA.

FIG. 12 is a photograph of a thin layer chromatogram showing results from PSI-BLAST search 1 candidate β-1,3 galactosyltransferases.

FIG. 13 is a table of pairwise amino acid sequence identity comparisons to GatA.

FIG. 14 is a photograph of a thin layer chromatogram showing LNT2 utilizing β-1,3 galactosyltransferases (comparison).

FIG. 15 is a table showing pairwise amino acid identity comparisons of newly discovered β-1,3 galactosyltransferase enzymes described herein with previously identified β-1,3 galactosyltransferases.

### DETAILED DESCRIPTION

The preferred route for efficient, industrial-scale synthesis of HMOs is through metabolic engineering of fermentable microbes, especially bacteria. This approach typically involves the construction of microbial strains expressing heterologous glycosyltransferases with desired specificities. In these strains, new metabolic pathways are often introduced, or existing pathways enhanced, to enable and increase production of regenerating nucleotide sugar pools for use as biosynthetic precursors in glycosyltransferase reactions (Bych et al., 2018; Dumon et al., 2004; Faijes et al., 2019; Mao et al., 2006; Petschacher and Nidetzky, 2016; Ruffing and Chen, 2006). These strains also need to express appropriate membrane transporters for both import of precursor sugars into the cell cytosol, and for export of products to the culture medium. A key aspect of the approach is selection of the particular heterologous glycosyltransferase, or combination of glycosyltransferases, to produce the desired HMO product. This choice, given that such enzymes can vary greatly in terms of kinetics, substrate specificity, affinity for donor and acceptor molecules, stability, solubility, and toxicity to the microbial host strain, can significantly affect final product yield and quality. Several glycosyltransferases derived from different bacterial species have previously been identified and characterized in terms of their ability to catalyze the biosynthesis of certain HMOs in E. coli host strains (Blixt et al., 1999; Drouillard et al., 2010; Dumon et al., 2006; Dumon et al., 2004; Li et al., 2008a; Li et al., 2008b; Zhu et al., 2021). However, there exists a continuing need to identify and characterize additional glycosyltransferases useful for biosynthesis or improved biosynthesis of particular HMOs in metabolically engineered microbes. The identification of additional glycosyltransferases with faster kinetics, greater affinity for nucleotide sugar donors and/or particular acceptor structures, greater stability within the heterologous microbial host, or higher specificity in producing desired molecules, has the potential to further improve HMO product yield and purity, and to make these molecules more broadly available for use as nutritional supplements and as therapeutics.

### β-1,3-Galactosyltransferases (β(1,3)GTs) for the Biosynthesis of β(1,3)-Galactosyl-Linked Oligosaccharides in Metabolically Engineered Microbes

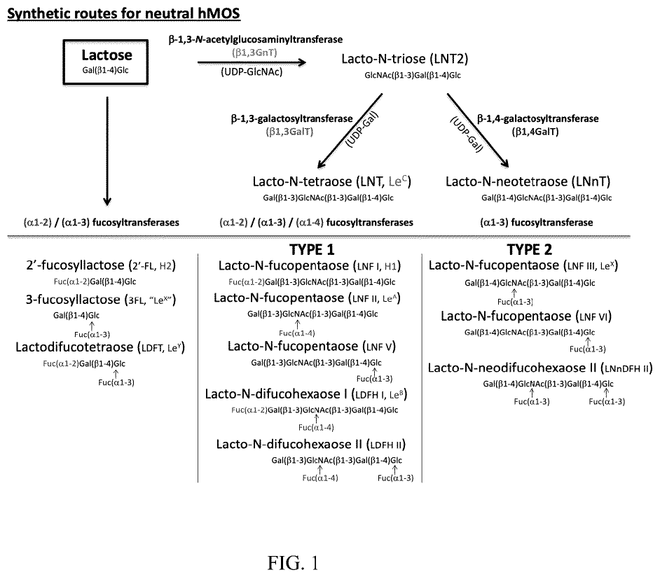

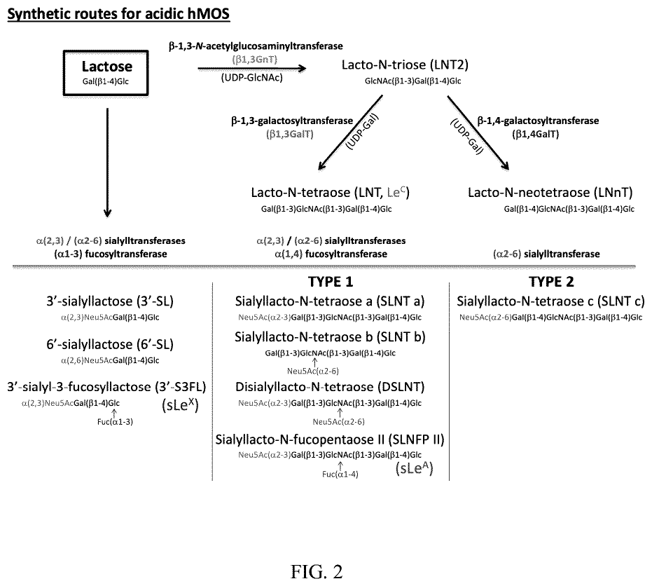

To this end, we have undertaken a candidate gene screening approach to identify new β-1,3-galactosyltransferases (β(1,3)GTs) for the synthesis of β(1,3)-galactosyl-linked oligosaccharides in metabolically engineered microbes. Of particular interest are new (β(1,3)GTs that are capable of forming the (Gal(β1-3)GlcNAc) “Type 1” motif as found in the human milk tetrasaccharide, lacto-N-tetraose (LNT). LNT is one of the most abundant oligosaccharides of human milk (Austin et al., 2016), and is thought to function with other HMOs as an important natural prebiotic, promoting the growth of beneficial commensal bacteria such as Bifidobacterium spp. in the infant gut, (James et al., 2016; Sakurama et al., 2013; Wada et al., 2008). LNT is not only itself a major individual component of the HMO mixture, but it forms the foundation of many higher molecular weight human milk oligosaccharides comprising the “Type 1” core, including but not limited to; lacto-N-fucopentaose I (LNF I), lacto-N-fucopentaose II (LNF II), lacto-N-fucopentaose V (LNF V), lacto-N-difucohexaose I (LDFH I), lacto-N-difucohexaose II (LDFH II), sialyllacto-N-tetraose a (SLNT-a), sialyllacto-N-tetraose b (SLNT-b), disialyllacto-N-tetraose (DSLNT) and sialyllacto-N-fucopentaose II (SLNFP II). FIG. 1 and FIG. 2 diagram synthetic schemes for syntheses of the most abundant neutral and acidic oligosaccharides (respectively) found in human milk. The Type 1 and Type 2 oligosaccharide classification groups are shown in each scheme.

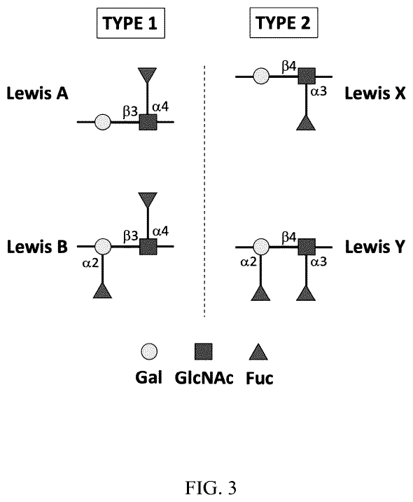

Type 1 and Type 2 glycan motifs exist not only in human milk oligosaccharides, but also within the structures of certain cell surface glycans in humans comprising antigens recognized under the “Lewis” typing system (Lloyd, 2000; Yuriev et al., 2005) (FIG. 3).

Individuals of Lewis A and Lewis B blood groups carry fucosylated glycans on the surface of red blood cells that comprise the Type 1 core. Lewis X and Lewis Y antigens, which incorporate the Type 2 core structure, are not found on blood cells but do exist on a few other cell types, for example certain epithelial cells such as gastric epithelium. Interestingly, Type 1 and Type 2 motifs, and “human-like” Lewis antigens, are additionally found in carbohydrate structures of the lipopolysaccharide found on the surface of a human bacterial pathogen, Helicobacter pylori, a gram-negative bacterium estimated to have colonized the stomachs of approximately 50% of humanity (Hooi et al., 2017). Helicobacter pylori colonization is usually chronic and typically benign. However sometimes the organism causes significant morbidity, precipitating conditions such as gastritis, stomach or duodenal ulcers, and even cancers (Kusters et al., 2006). One intriguing aspect of H. pylori biology is its avoidance of host immune responses during chronic colonization, and one part of this seems to be its ability to adapt genetically to alter the carbohydrate content of its surface lipopolysaccharide to match/mimic the host's Lewis antigen type, i.e., to become more like “self”, and thus evade host immune surveillance. One study (Pohl et al., 2009) highlighted genetic changes in a putative and defective β1,3) galactosyltransferase gene found in the Lewis B negative Helicobacter pylori HP1 as the strain switched to a Lewis B positive phenotype following 8 months of in vivo selection in Lewis B positive transgenic mice. The wild type, putative and defective β(1,3)GT gene of strain HP1 (itself a homolog of a putative and defective, “lipopolysaccharide biosynthesis gene” (JHP0563) from H. pylori strain J99) contained a frameshift that destroyed its reading frame, whereas the Lewis B positive Helicobacter pylori HP1 variant that emerged after in vivo selection (clone 03-270) had mutated (by inserting two nucleotides into the defective JHP0563 variant β(1,3)GT gene) to restore the open reading frame (JHP0563 variant, clone 03-270. SEQ ID NO: 15, (Pohl et al., 2009)).

Encouraged by this evidence that the restored HP β(1,3)GT gene may thus encode an active β(1,3) galactosyltransferase, we used the JHP0563 protein sequence to probe, using BLAST homology searches (Altschul et al., 1990), several complete Helicobacter pylori genomes located in public DNA sequence databases, looking for full-length, intact, homologs of JHP0563 that might represent authentic wild type β-1,3-galactosyltransferase genes. Helicobacter pylori strain P12 contained such a homolog. We named this putative β-1,3-galactosyltransferase enzyme “GatA”, whose amino acid sequence is presented as SEQ ID NO: 1. GatA is represented in public sequence databases under accession #ACJ07781.1

Similar to Helicobacter pylori, lipopolysaccharide (LPS) also comprises the outermost layer of the Escherichia coli cell envelope. The external surface of this envelope LPS in E. coli is decorated with a highly diverse polysaccharide called the “0” antigen, whose precise composition and structure varies dramatically between different E. coli strains. 181 distinct “0” antigen variants have been formally defined (Liu et al., 2020). In contrast to H. pylori, E. coli “0” antigens are usually highly immunogenic, however it is thought that their extreme diversity offers selective advantages in particular niches for individual strain clones (Wang et al., 2010), and thus LPS variants are maintained. The enteropathogenic E. coli 055:H7 strain's “0” antigen comprises a repeating pentasaccharide structure featuring the familiar Gal(β1-3)GlcNAc motif. The E. coli 055:H7 β-1,3-galactosyltransferase enzyme responsible for formation of this structure, WbgO, has been identified and characterized (Liu et al., 2009), and the amino acid sequence of WbgO (accession #YP_003500090.1) is presented as SEQ ID NO: 2.

The extraintestinal pathogenic E.coli strain O7:K1 “O” antigen is also a repeating pentasaccharide structure featuring the Gal(β1-3)GlcNAc motif. The E. coli O7:K1 3-1,3-galactosyltransferase enzyme responsible for formation of this structure, WbbD, has been identified and characterized (Riley et al., 2005), and the amino acid sequence of WbbD (accession #YP_006144407.1) is presented as SEQ ID NO: 3.

### Example 1: Engineering E. coli to Generate Host Strains for the Production of Lacto-N-Tetraose (LNT)

The E. coli K12 prototroph, W3110, was chosen as the parent background for LNT biosynthesis. This strain had previously been modified at the ampC locus by the introduction of a tryptophan-inducible P_{trpB}-cI+ repressor construct (McCoy and Lavallie, 2001), enabling convenient, controllable production of recombinant proteins from the phage λ P_{L} promoter (Sanger et al., 1982) through induction with millimolar concentrations of tryptophan (Mieschendahl et al., 1986). The strain GI724, an E. coli W3110 derivative containing the tryptophan-inducible P_{trpB}-cI+ repressor construct in ampC, was used at the basis for further E. coli strain manipulations

Biosynthesis of LNT requires the generation of an enhanced cellular pool of lactose. This enhancement was achieved in strain GI724 through several manipulations of the chromosome using k Red recombineering (Court et al., 2002) and generalized P1 phage transduction (Thomason et al., 2007). The ability of the E. coli host strain to accumulate intracellular lactose was first engineered by simultaneous deletion of the endogenous β-galactosidase gene (lacZ) and the lactose operon repressor gene (lacI). During construction of this deletion, the constitutive lacIq promoter was placed immediately upstream of the lactose permease gene, lacY. The modified strain thus maintains its ability to transport lactose from the culture medium (via LacY), but is deleted for the wild-type copy of the IacZ (β-galactosidase) gene responsible for lactose catabolism. An intracellular lactose pool is therefore created when the modified strain is cultured in the presence of exogenous lactose.

An optional or additional modification useful for increasing the cytoplasmic pool of free lactose (and hence the final yield of LNT) is the incorporation of a lacA mutation. LacA is a lactose acetyltransferase that is only active when high levels of lactose accumulate in the E. coli cytoplasm. High intracellular osmolarity (e.g., caused by a high intracellular lactose pool) can inhibit bacterial growth, and E. coli has evolved a mechanism for protecting itself from high intra cellular osmolarity caused by lactose by “tagging” excess intracellular lactose with an acetyl group using LacA, and then actively expelling the acetyl-lactose from the cell (Danchin, 2009). Production of acetyl-lactose in E. coli engineered to produce human milk oligosaccharides is therefore undesirable: it reduces overall yield. Moreover, acetyl-lactose is a side product that complicates oligosaccharide purification schemes. The incorporation of a lacA mutation resolves these problems, as carrying a deletion of the lacA gene renders the bacterium incapable of synthesizing acetyl-lactose.

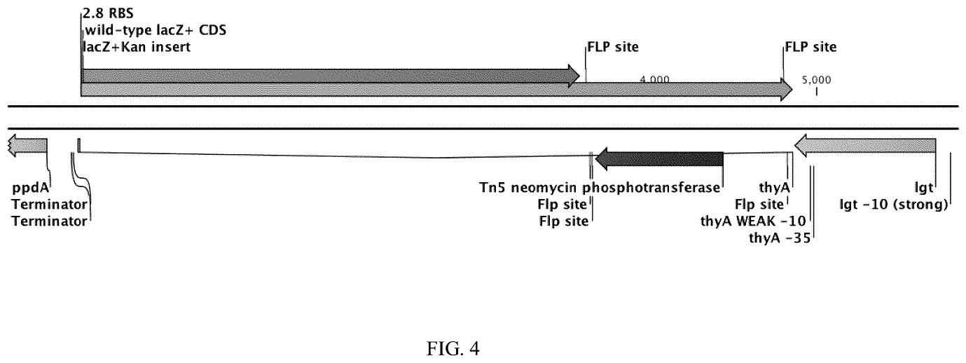

A thyA (thymidylate synthase) mutation was introduced by almost entirely deleting the thyA gene and replacing it by an inserted functional, wild-type, but promoter-less E. coli lacZ⁺ gene carrying the 2.8 ribosome binding site (ΔthyA::(2.8RBS lacZ⁺,kan^{r}). X Red recombineering (Court et al., 2002) was used to perform the construction. FIG. 4 illustrates the new configuration of genes thus engineered at the thyA locus.

Genomic DNA sequence surrounding the lacZ+ insertion into the thyA region is set forth in SEQ ID NO: 4.

The thyA defect can be complemented in trans by supplying a wild-type thyA gene on a multicopy plasmid (Belfort et al., 1983). This complementation is used herein as a means of plasmid maintenance (eliminating the need for a more conventional antibiotic selection scheme to maintain plasmid copy number).

The genotype of strain E680 is given below. E680 incorporates all the changes discussed above and is a host strain suitable for the production of lacto-N-tetraose (LNT).

F′402 proA+B+, PlacIq-lacY, Δ(lacI-lacZ)158, ΔlacA398 araC, Δgpt-mhpC, ΔthyA::(2.8RBS lacZ+, KAN), rpoS+, rph+, ampC::(Ptrp T7g10 RBS-λcI+, CAT).

### Example 2. Production of lacto-N-tetraose (LNT) in E. coli

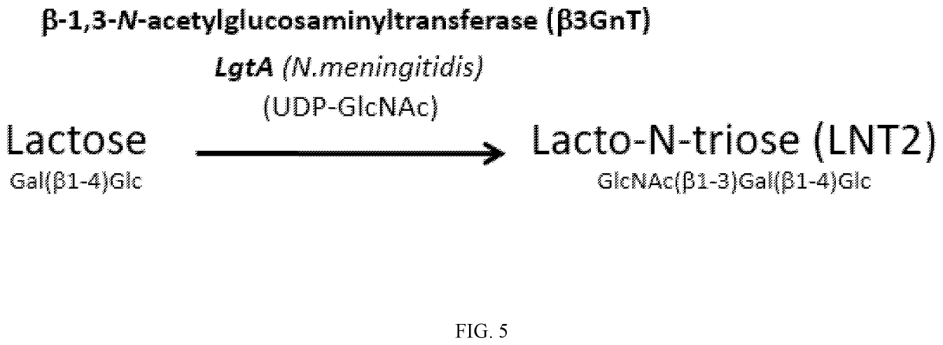

The first step in the synthesis (from a lactose precursor) of lacto-N-tetraose (LNT) is the addition of a β(1,3)N-acetylglucosamine residue to lactose, utilizing a heterologous β(1,3)-N-acetylglucosaminyltransferase (β1,3GnT) to form lacto-N-triose 2 (LNT2). FIG. 5 illustrates this reaction.

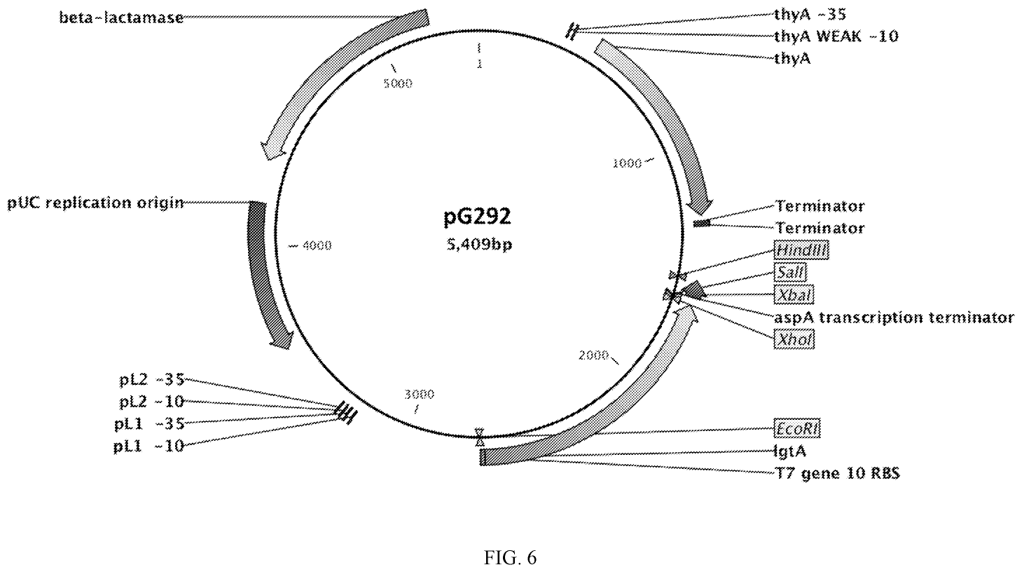

The plasmid pG292 (ColE1, thyA+, bla+, P_{L}-lgtA) (SEQ ID NO: 5, FIG. 6) carries the lgtA β(1,3)-N-acetylglucosaminyltransferase gene of Neisseria meningitidis (Blixt et al., 1999) and can direct the production of LNT2 in E. coli strain E680 under appropriate culture conditions. See SEQ ID NO: 5 pG292.

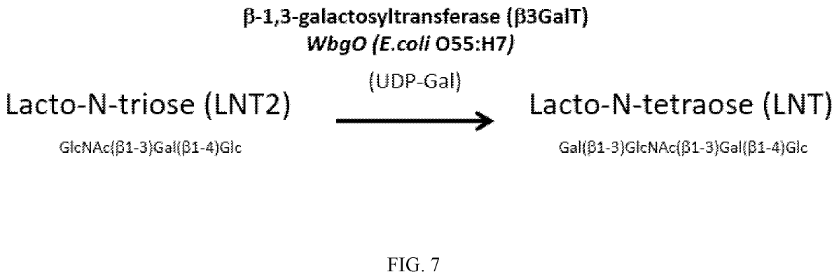

FIG. 7 illustrates the conversion of LNT2 to LNT by a (1,3)galactosyltransferase, for example WbgO.

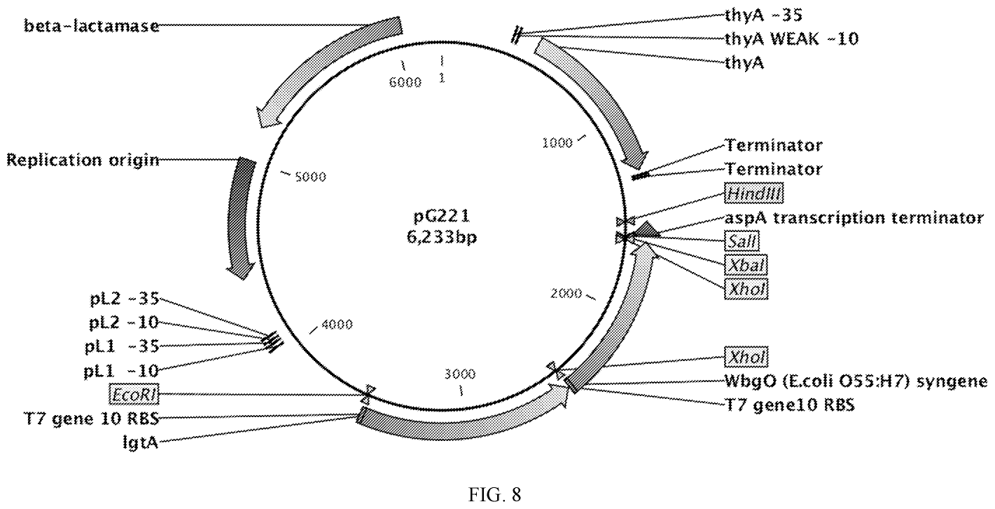

pG221 (ColE1, thyA+, bla+, P_{L}-1gtA-wbgO) (SEQ ID NO: 6, FIG. 8) is a derivative of pG292 that carries both the IgtA β(1,3)-N-acetylglucosaminyltransferase gene of N. meningitidis and the wbgO β(1,3)-galactosyltransferase gene of E. coli 055:H7 (arranged on the plasmid as a two-gene operon). pG221 directs the production of LNT in E. coli strain E680 under appropriate culture conditions. See SEQ ID NO: 6 pG221.

The addition of tryptophan to lactose-containing growth medium of cultures of either of the E680-derivative strains transformed with plasmids pG292 or pG221 leads, for each particular E680/plasmid combination, to activation of the host E. coli tryptophan utilization repressor TrpR, subsequent repression of P_{trpB}, and a consequent decrease in cytoplasmic cI levels, which results in a de-repression of P_{L}, expression of IgtA or IgtA+wbgO respectively, and production of LNT2 or LNT2 and LNT, respectively.

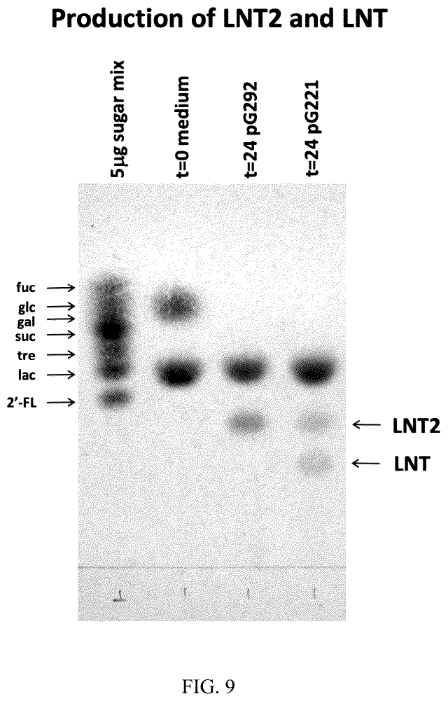

For LNT2 or LNT production in small scale laboratory cultures (<100 ml), strains were grown at 30° C. to early exponential phase in IMC medium (M9 salts, 0.5% glucose, 0.4% casaminoacids, and lacking both thymidine and tryptophan). Lactose was then added to a final concentration of 0.5 or 1%, along with tryptophan (200 μM final) to induce expression of the respective glycosyltransferases, driven from the P_{L} promoter. At the end of the induction period (˜24 h), TLC analysis was performed on aliquots of cell-free culture medium. FIG. 9 shows a thin layer chromatogram of culture medium samples taken from small scale E. coli cultures, and demonstrating synthesis of LNT2 and LNT (utilizing induced, lactose-containing cultures of E680 transformed with pG292 or pG221, respectively).

### Example 3. Comparing Known β-1,3-Galactosyltransferase Enzymes WgbO and WbbD with the Putative β-1,3-Galactosyltransferase “GatA” for Production of Lacto-N-Tetraose (LNT) in E. coli

To compare the ability of putative β-1,3-galactosyltransferase “GatA” (from Helicobacter pylori P12) with known β-1,3-galactosyltransferases WbgO (from E. coli 055:H7) and WbbD (from E. coli 07:K1) for the synthesis of LNT in engineered E. coli K-12 host strain E680, two additional plasmids were constructed; pG293 (SEQ ID NO: 7) and pG294 (SEQ ID NO: 8). In these two plasmids, the WbgO coding sequence present in plasmid pG221 was replaced precisely by DNA sequences encoding WbbD and GatA, respectively. See SEQ ID NO: 7 pG293 and SEQ ID NO: 8 pG294.

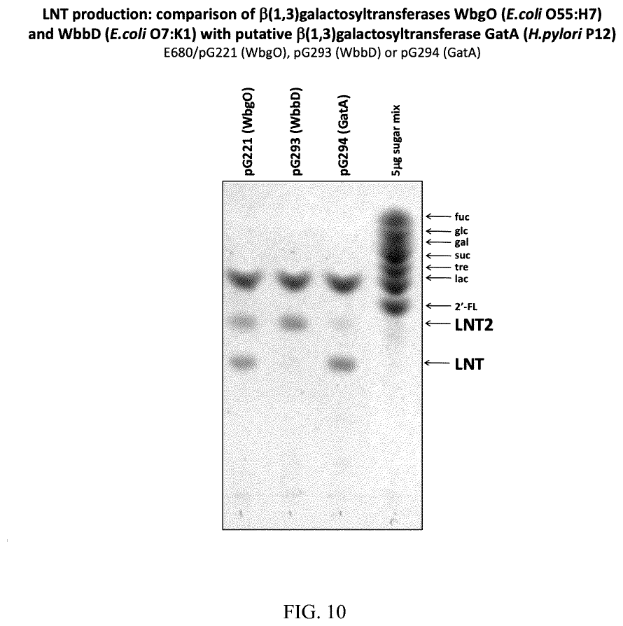

For LNT production at small scale (5 ml), cultures comprising host strain E680 transformed with either pG221 (WbgO), pG293 (WbbD) or E294 (GatA) were grown at 30° C. to early exponential phase in IMC medium (M9 salts, 0.5% glucose, 0.4% casaminoacids, and lacking both thymidine and tryptophan). Lactose was then added to a final concentration of 0.5%, along with tryptophan (200 μM final) to induce expression of β(1,3)-N-acetylglucosaminyltransferase LgtA along with the respective β-1,3-galactosyltransferase, both driven from the P_{L} promoter. At the end of the induction period (˜24 h), TLC analysis was performed on aliquots of cell-free culture medium. FIG. 10 shows a thin layer chromatogram of culture medium samples taken from small scale E. coli cultures and demonstrating synthesis of both LNT2 and LNT (utilizing induced, lactose-containing cultures of E680 transformed with pG221, pG293 and pG294). As can be seen pG221, expressing WbgO the known β-1,3-galactosyltransferase control, produced LNT as expected. pG294 expressing GatA, the putative β-1,3-galactosyltransferase from H. pylori P12, also produced LNT, for the first time confirming that GatA is indeed a β-1,3-galactosyltransferase. Interestingly the conversion of LNT2 to LNT looked more complete with GatA than it did with WbgO. Unexpectedly, pG293 expressing WbbD, produced only a trace of LNT, if any at all.

### Example 4. Searching Public DNA Sequence Databases for Additional Candidate β-1,3-Galactosyltransferase Enzymes

We used the amino acid sequence of GatA as a query for the database search algorithm PSI-BLAST (Position Specific Iterated Basic Local Alignment Search Tool) in an effort to identify additional candidate β-1,3-galactosyltransferase enzymes. To execute a PSI-BLAST search, a list of closely related proteins is created based on a query sequence. These proteins are then combined into a general profile sequence, which summarizes significant motifs present in these sequences. This profile is then used as a query to identify a larger group of proteins, and the process is repeated to generate an even larger group of candidates (Altschul et al., 1990; Altschul et al., 1997).

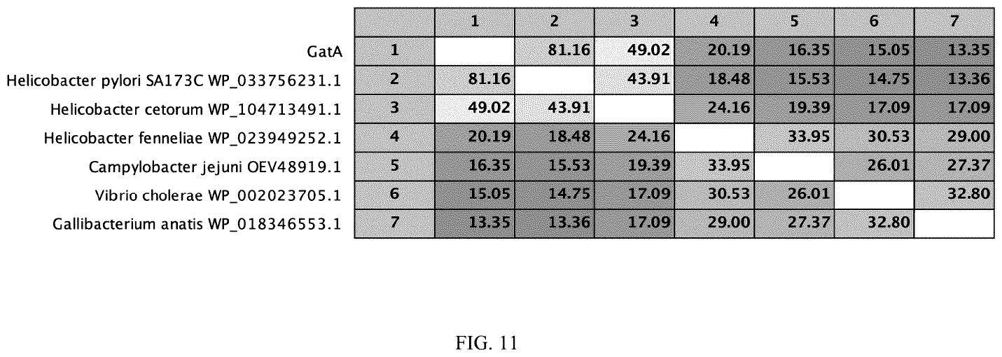

We used the GatA amino acid sequence as a query for three search iterations in an initial PSI-BLAST screen. This approach yielded a group of several hundred candidates that was winnowed down by removing all hits to eukaryotes and archaea, hits with alignment lengths to GatA of less than 200 amino acids, hits to Helicobacter pylori sequences less than 350 amino acids in alignment length, hits to candidates with % identity to GatA of less than 13%, and by focusing on hits from pathogenic species. We selected 6 predicted β(1,3)GT candidates from this first PSI-BLAST screen, with homologies to GatA ranging from 13-81% at the amino acid level, for experimental validations. FIG. 11 presents a table of pairwise amino acid sequence identity comparisons to GatA for these six β(1,3)GT candidates. Species source, accession number, SEQ ID NO, and a candidate identifier for each are included in Table 1.

TABLE 1

| Candidate | | | SEQ ID |
|---|---|---|---|
| identifier | Species source | Accession # | NO: |
| GatA | Helicobacter pylori P12 | ACJ07781.1 | 1 |
| Hp2 | Helicobacter pylori SA173C | WP_033756231.1 | 9 |
| Hc1 | Helicobacter cetorum | WP_104713491.1 | 10 |
| | 138563_8 | | |
| Hf1 | Helicobacter fenneliae | WP_023949252.1 | 11 |
| Cj1 | Campylobacter jejuni | OEV48919.1 | 12 |
| Vc1 | Vibrio cholerae | WP_002023705.1 | 13 |
| Ga1 | Gallibacterium anatis | WP_018346553.1 | 14 |

Coding regions for each of the 6 candidate β(1,3)GT genes were cloned by standard molecular biological techniques (Green et al., 2012) into expression plasmid pG221, with the WbgO coding sequence in pG221 being precisely replaced with the coding sequence of each candidate.

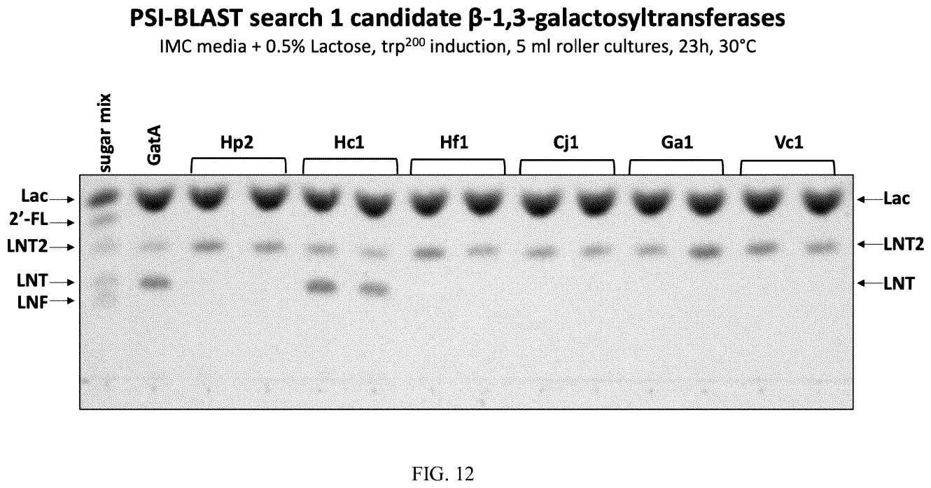

E680-derived E. coli strains harboring the six β(1,3)GT candidate gene expression plasmids were analyzed (in duplicate) in small-scale experiments. Strains were grown in IMC media (M9 salts containing glucose at 0.5% and casamino acids at 0.4%, and lacking thymidine), to early exponential phase at 30° C. Lactose was then added to a final concentration of 0.5%, and tryptophan (200 μM) was added to induce expression of each candidate from the P_{L}promoter. At the end of the induction period (˜23 h) aliquots of clarified media from each strain culture were analyzed for the presence of LNT2 and LNT by thin layer chromatography (TLC). As shown in FIG. 12, a control strain expressing LgtA and GatA showed, as expected, biosynthesis of both LNT2 and LNT. Each of the β(1,3)GT candidate cultures also showed production of LNT2. However, the Hc1 β(1,3)GT candidate culture also produced LNT, indicating for the first time that Helicobacter cetorum WP_104713491.1 is a 3-1,3-galactosyltransferase. The fact that only one of the six candidates tested was able to synthesize LNT in our engineered E. coli strain indicates the novelty and uniqueness of our findings.

### Example 5. Searching Public DNA Sequence Databases for Additional Candidate β-1,3-Galactosyltransferase Enzymes

We conducted a second PSI-BLAST screen looking for additional candidate 3-1,3-galactosyltransferases. For this query in this second screen, we used a profile that was derived from a multiple sequence alignment of four known β-1,3-galactosyltransferase enzymes, i.e.;
1. GatA (SEQ ID NO: 1 from this study, ACJ07781.1)
2. Hc1 (SEQ ID NO: 10 from this study, WP_104713491.1)
3. jhp0563 from Helicobacter pylori strain 03-270 (from (Pohl et al., 2009), JQ002580.1, SEQ ID NO: 15)
4. Sequence 1 Helicobacter pylori (strain unknown) R (1,3)GT from U.S. Pat. No. 6,974,687, SEQ ID NO: 16

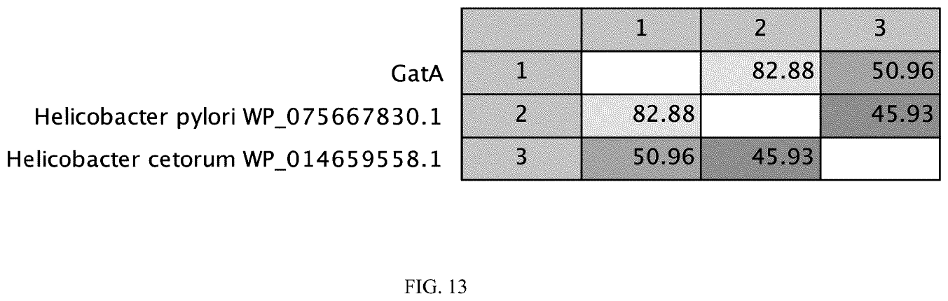

We used the above profile as the query for four search iterations in this second PSI-BLAST screen. The search yielded a group of several hundred candidates that was winnowed down again by removing all hits to eukaryotes and archaea, hits with alignment lengths less than 200 amino acids, hits to Helicobacter pylori sequences less than 325 amino acids in alignment length, hits to candidates with % identity to GatA less than 15%, and by focusing on hits from pathogenic species. We selected just two predicted β(1,3)GT candidates from this screen. FIG. 13 presents a table of pairwise amino acid sequence identity comparisons to GatA of these two β(1,3)GT candidates. Species source, accession number, SEQ ID NO, and a candidate identifier for each are included in Table 2.

TABLE 2

| Candidate | | | SEQ ID |
|---|---|---|---|
| identifier | Species source | Accession # | NO: |
| GatA | Helicobacter pylori P12 | ACJ07781.1 | 1 |
| Hp3 | Helicobacter pylori H9 | WP_075667830.1 | 17 |
| Hc2 | Helicobacter cetorum | WP_014659558.1 | 18 |
| | MIT 99-5656 | | |

Coding regions for the 2 additional candidate β(1,3)GT genes (Hp3 and Hc2) were cloned by standard molecular biological techniques (Green et al., 2012) into expression plasmid pG221, with the WbgO coding sequence in pG221 being precisely replaced with the coding sequence of each candidate.

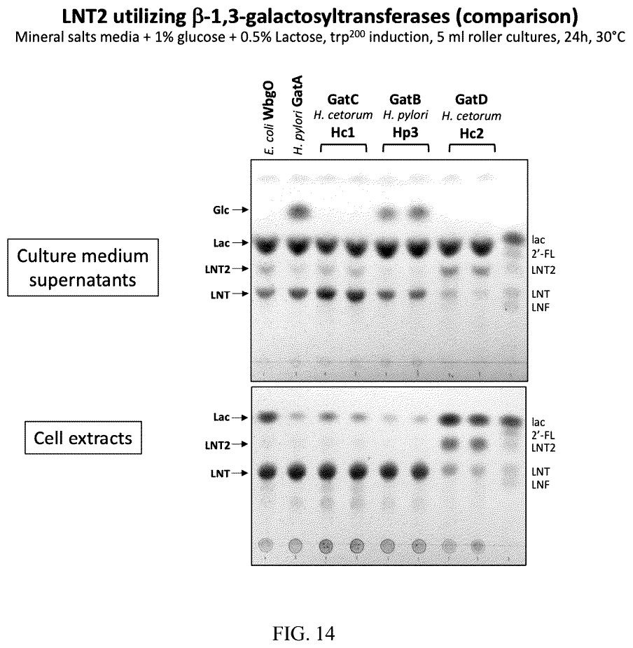

E680-derived E. coli strains harboring the 2 additional β(1,3)GT candidate gene expression plasmids were analyzed (in duplicate) in small-scale experiments. Strains were grown in a mineral salts selective media (containing glucose at 1%, but lacking thymidine), to early exponential phase at 30° C. Lactose was then added to a final concentration of 0.5%, and tryptophan (200 μM) was added to induce expression of each candidate from the P_{L} promoter. At the end of the induction period (˜24 h) aliquots of clarified media from each strain culture were analyzed for the presence of LNT2 and LNT by thin layer chromatography (TLC). The presence of LNT2 and LNT inside the cells was also examined by additionally running aliquots of soluble heat extracts of candidate strain cell pellets on the TLC (treatment at 95° C., 10 minutes). The new candidates were compared on the TLC with a strain containing WbgO, a strain containing GatA, and a strain containing Hc1 from the first PSI-BLAST screen. As shown in FIG. 14, the control strains expressing LgtA and GatA, and LgtA and Hc1 showed, as expected, biosynthesis of both LNT2 and LNT. Each of the cultures expressing the two new β(1,3)GT candidates (Hp3 and Hc2) also showed production of both LNT2 and LNT, for the first time showing that both of these two enzymes are indeed β-1,3-galactosyltransferases. Hp3 utilized the available LNT2 better than all other enzymes tested, and Hc2 produced the lowest level of LNT overall.

In summary, we have used a directed screening approach to identify and characterize four new bacterial LNT2-accepting β-1,3-galactosyltransferases. We named these enzymes GatA, GatB, GatC and GatD. Table 3 lists these names along with previous candidate identifiers, source organisms and strains, database accession numbers, and SEQ ID NOs.

TABLE 3

| Pro- | Previous | | | SEQ |
|---|---|---|---|---|
| tein | candidate | | | ID |
| name | identifier | Species source | Accession # | NO: |
| GatA | GatA | Helicobacter pylori P12 | ACJ07781.1 | 1 |
| GatB | Hp3 | Helicobacter pylori H9 | WP_075667830.1 | 17 |
| GatC | Hc1 | Helicobacter cetorum | WP_104713491.1 | 10 |
| | | 138563_8 | | |
| GatD | Hc2 | Helicobacter cetorum | WP_014659558.1 | 18 |
| | | MIT 99-5656 | | |

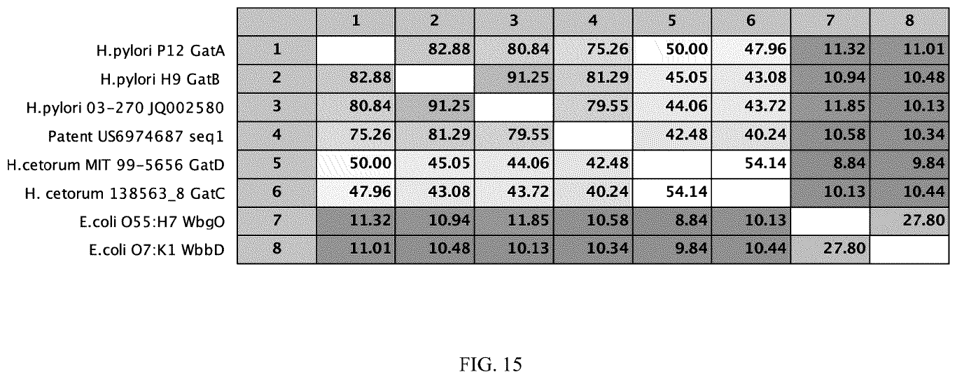

FIG. 15 shows a pairwise amino acid identity comparison of the four newly discovered LNT2-accepting β-1,3-galactosyltransferases of this work, GatA, GatB, GatC and GatD, with previously identified β-1,3-galactosyltransferases mentioned above.

We have shown that these newly discovered β-1,3-galactosyltransferases are useful in the production of LNT in small scale microbial cultures, and thus they will be useful in the production at large scale of LNT and a variety of other Type 1 human milk oligosaccharides to supply demand for these important molecules as nutritional supplements and therapeutics.

### BIBLIOGRAPHY

Altschul S F, et al., (1990) Basic local alignment search tool. J Mol Biol 215:403-410.
Altschul S F, et al., (1997) Gapped BLAST and PSI-BLAST: a new generation of protein database search programs. Nucleic acids research 25:3389-3402.
Austin S, et al., (2016) Temporal Change of the Content of 10 Oligosaccharides in the Milk of Chinese Urban Mothers. Nutrients 8.
Belfort M, et al., (1983) Primary structure of the Escherichia coli thyA gene and its thymidylate synthase product. Proc Natl Acad Sci USA 80:4914-4918.
Blixt 0, et al., (1999) High-level expression of the Neisseria meningitidis lgtA gene in Escherichia coli and characterization of the encoded N-acetylglucosaminyltransferase as a useful catalyst in the synthesis of GlcNAc beta 1-->3Gal and GalNAc beta 1-->3Gal linkages. Glycobiology 9:1061-1071.
Bode L and Jantscher-Krenn E (2012) Structure-function relationships of human milk oligosaccharides. Adv Nutr 3:383S-391S.
Bych K, et al., (2018) Production of HMOs using microbial hosts—from cell engineering to large scale production. Curr Opin Biotechnol 56:130-137.
Chaturvedi P, et al., (1997) Milk oligosaccharide profiles by reversed-phase HPLC of their perbenzoylated derivatives. Anal Biochem 251:89-97.
Cheng L, et al., (2020) More than sugar in the milk: human milk oligosaccharides as essential bioactive molecules in breast milk and current insight in beneficial effects. Crit Rev Food Sci Nutr:1-17.
Court D L, et al., (2002) Genetic engineering using homologous recombination. Annu Rev Genet 36:361-388.
Danchin A (2009) Cells need safety valves. Bioessays 31:769-773.
Drouillard S, et al., (2010) Efficient synthesis of 6′-sialyllactose, 6,6′-disialyllactose, and 6′-KDO-lactose by metabolically engineered E. coli expressing a multifunctional sialyltransferase from the Photobacterium sp. JT-ISH-224. Carbohydr Res 345:1394-1399.
Dumon C, et al., (2006) Production of Lewis x tetrasaccharides by metabolically engineered Escherichia coli. Chembiochem 7:359-365.
Dumon C, et al., (2004) Assessment of the two Helicobacter pylori alpha-1,3-fucosyltransferase ortholog genes for the large-scale synthesis of LewisX human milk oligosaccharides by metabolically engineered Escherichia coli. Biotechnol Prog 20:412-419.
Faijes M, et al., (2019) Enzymatic and cell factory approaches to the production of human milk oligosaccharides. Biotechnol Adv.
Gnoth M J, et al., (2000) Human milk oligosaccharides are minimally digested in vitro. J Nutr 130:3014-3020.
Green M R, Sambrook J and Sambrook J Mc (2012) Molecular cloning: a laboratory manual, Cold Spring Harbor Laboratory Press, Cold Spring Harbor, N.Y.
Hooi J K Y, et al., (2017) Global Prevalence of Helicobacter pylori Infection: Systematic Review and Meta-Analysis. Gastroenterology 153:420-429.
James K, et al., (2016) Bifidobacterium breve UCC2003 metabolises the human milk oligosaccharides lacto-N-tetraose and lacto-N-neo-tetraose through overlapping, yet distinct pathways. Sci Rep 6:38560.
Kunz C, et al., (2000) Oligosaccharides in human milk: structural, functional, and metabolic aspects. Annu Rev Nutr 20:699-722.
Kusters J G, et al., (2006) Pathogenesis of Helicobacter pylori infection. Clinical microbiology reviews 19:449-490.
Li M, et al., (2008a) Characterization of a novel alpha1,2-fucosyltransferase of Escherichia coli 0128:b12 and functional investigation of its common motif. Biochemistry 47:378-387.
Li M, et al., (2008b) Identification of a new alpha1,2-fucosyltransferase involved in O-antigen biosynthesis of Escherichia coli 086:B7 and formation of H-type 3 blood group antigen. Biochemistry 47:11590-11597.
Liu B, et al., (2020) Structure and genetics of Escherichia coli O antigens. FEMS Microbiol Rev 44:655-683.
Liu X-w, et al., (2009) Characterization and synthetic application of a novel beta1,3-galactosyltransferase from Escherichia coli 055:H7. Bioorg Med Chem 17:4910-4915.
Lloyd K O (2000) The chemistry and immunochemistry of blood group A, B, H, and Lewis antigens: past, present and future. Glycoconjugate Journal 17:531-541.
Mao Z, et al., (2006) Engineering the E. coli UDP-glucose synthesis pathway for oligosaccharide synthesis. Biotechnol Prog 22:369-374.
McCoy J and Lavallie E (2001) Expression and purification of thioredoxin fusion proteins. Curr Protoc Mol Biol Chapter 16:Unit16 18.
Mieschendahl M, et al., (1986) A novel prophage independent TRP regulated lambda PL expression system. Nature Biotechnology 4:802-808.
Newburg D S and Walker W A (2007) Protection of the neonate by the innate immune system of developing gut and of human milk. Pediatr Res 61:2-8.
Petschacher B and Nidetzky B (2016) Biotechnological production of fucosylated human milk oligosaccharides: prokaryotic fucosyltransferases and their use in biocatalytic cascades or whole cell conversion systems. J Biotechnol.
Pohl M A, et al., (2009) Host-dependent Lewis (Le) antigen expression in Helicobacter pylori cells recovered from Leb-transgenic mice. Journal of Experimental Medicine 206:3061-3072.
Ray C, et al., (2019) Human Milk Oligosaccharides: The Journey Ahead. Int J Pediatr 2019:2390240.
Riley J G, et al., (2005) The wbbD gene of E. coli strain VW187 (07:K1) encodes a UDP-Gal: GlcNAc{alpha}-pyrophosphate-R {beta}1,3-galactosyltransferase involved in the biosynthesis of 07-specific lipopolysaccharide. Glycobiology 15:605-613.
Rudloff S and Kunz C (2012) Milk oligosaccharides and metabolism in infants. Adv Nutr 3:3985-405S.
Ruffing A and Chen R R (2006) Metabolic engineering of microbes for oligosaccharide and polysaccharide synthesis. Microb Cell Fact 5:25.
Sakurama H, et al., (2013) Lacto-N-biosidase encoded by a novel gene of Bifidobacterium longum subspecies longum shows unique substrate specificity and requires a designated chaperone for its active expression. J Biol Chem 288:25194-25206.
Sanger F, et al., (1982) Nucleotide sequence of bacteriophage lambda DNA. J Mol Biol 162:729-773.
Thomason L C, et al., (2007) E. coli genome manipulation by P1 transduction. Curr Protoc Mol Biol Chapter 1:Unit 1.17.
Wada J, et al., (2008) Bifidobacterium bifidum lacto-N-biosidase, a critical enzyme for the degradation of human milk oligosaccharides with a type 1 structure. Appl Environ Microbiol 74:3996-4004.
Wang L, et al., (2010) The variation of O antigens in gram-negative bacteria. Subcell Biochem 53:123-152.
Yuriev E, et al., (2005) Three-dimensional structures of carbohydrate determinants of Lewis system antigens: implications for effective antibody targeting of cancer. Immunology and cell biology 83:709-717.
Zhu Y, et al., (2021) Metabolic Engineering of Escherichia coli for Efficient Biosynthesis of Lacto-N-tetraose Using a Novel β-1, 3-Galactosyltransferase from Pseudogulbenkiania ferrooxidans. Journal of Agricultural and Food Chemistry 69:11342-11349.

B-1,3-galactosyltransferase sequences
H. pylori P12 GatA (3GalT) ACJ07781
>GatA_(3GalT)_ACJ07781.1 lipopolysaccharide biosynthesis protein
[Helicobacter pylori P12].
SEQ ID NO: 1
MIGVYIISLKESQRRLDTEKLVSESNEKFKGRCVFQIFDAISPKHEDFEKFVQELYDAQS
MLKSDWFHSDYCYQELLPREFGCYLGHYFLWKECVKTNQPVVILEDDVALESNEMQALED
CLKSPFDFVRLYGHYWGGHKTNLCALPIYTEAEVPIENHEVTPPPPNPARDTQQDFIIET
QQDPKEPSDPCKIAPQKISFNQVVFKKIKRKLNRFIGSILARTEVYKNVVAKYDDLTKKY
DDLTKKYDELTGKYESLLAKETNIKETFWERRADNEKEALFLEHFYLTSVYVATTAGYYL
TPKGAKTFIEATERFKIIEPVDMFMNNPTYHDVANFTYLPCPVSLNKHAFNSTIQNAKKP
DISLKSPKKSYFDNLFYDQLNTKKCLRAFHKYSKQYAPLKTPKEI
E. coli WbgO YP_003500090
>WbgO_YP_003500090 putative glycosyltransferase WbgO
SEQ ID NO: 2
[Escherichia coli 055: H7 str. CB9615].
MIIDEAESAESTHPVVSVILPVNKKNPFLDEAINSILSQTFSSFEIIIVANCCTDDFYNE
LKHKVNDKIKLIRTNIAYLPYSLNKAIDLSNGEFIARMDSDDISHPDRFTKQVDELKNNP
YVDVVGTNAIFIDDKGREINKTKLPEENLDIVKNLPYKCCIVHPSVMERKKVIASIGGYM
FSNYSEDYELWNRLSLAKIKFQNLPEYLFYYRLHEGQSTAKKNLYMVMVNDLVIKMKCFF
LTGNINYLFGGIRTIASFIYCKYIK
E. coli WbbD YP 006144407
>WbbD_YP_006144407 UDP-Gal: GlcNAc alpha-pyrophosphate-R beta 1, 3-
galactosyltransferase [Escherichia coli 07: K1 str. CE10].
SEQ ID NO: 3
MSDDTPKFSVLMAIYIKDSPLFLSEALQSIYKNTVAPDEVIIIRDGKVTSELNSVIDSWR
RYLNIKDFTLEKNMGLGAALNFGLNQCMHDLVIRADSDDINRTNRFECILDFMTKNGDVH
ILSSWVEEFEFNPGDKGIIKKVPSRNSILKYSKNRSPFNHPAVAFKKCEIMRVGGYGNEY
LYEDYALWLKSLANGCNGDNIQQVLVDMRESKETAKRRGGIKYAISEIKAQYHFYRANYI
SYQDFIINIITRIFVRLLPTSFRGYIYKKVIRRFL
thy A 2.8RBS lacZ
>E680_thyA_2.8RBS_lacZ, KAN Escherichia coli str. K-12
SEQ ID NO: 4
TCACAGGTTGAATCCTGTCACGCTATAGCTGGCATTCACCACGGTTTGCGGTTCAGACTT
ACTGGCAGCACGCATATTAACCGTCAACACCGGCGAGAAGCCGCTGACATCCTGACGTAC
GACCTGAAAAGTGTCGATAATGATGGCATCCGGATTAGTGACTTTATCCCAGCCCTTACC
TTCACAGGATGTCGCACCGCGTAGCGTTTCCAGCACATGCTCCTTCAGACGAAATCCAAT
CTGGTCGGACTCTTTTACCGGTTCGCGATCCCAGATACCGTTACTGTTCGCATCCCACTG
CACAATGACACAGTCACCCTGTCCGACAATTTCCAGCCCTTCGCCGGTACAGATGCCATG
ACAATAACCCGCCCTCTGGAGATGCTTCGCGACGGTAAATACCCGCAGCCAGATTTCATC
TTCCAGCGCCAGCTTACGGGTGCTCGTTAAACTTTCACGCTGTAACGCAGGCAGAAAGCG
TGCCGCCCCCAGCAACAATACGCTACTGATCGCCATAGCAATCAACACTTCCAGCAGAGA
AAAACCTTGCTCTTTTACAGGCATCCTTCTGTTTCTCCTTGCTGACAAAGCCGGAGTCTT
CCCCACGGCGAAACCACCAGCCACCACTCGCCCGTTGAGTTTTTGAAGCGAATATGCCCG
GCCCATGCGGTATTGCGCAGGCCAAAGAAAGCAAGCGAAGGTGTCAGGTCGCTCATTTCG
ACTTCGGGCCAGCGTGGCACAAAGACCAATGGTGAACTGCCATGACAGGTATTGGCCCCA
GCAGCGGAACTCACAAGGCACCATAACGTCCCCTCCCTGATAACGCTGATACTGTGGTCG
CGGTTATGCCAGTTGGCATCTTCACGTAAATAGAGCAAATAGTCCCGCGCCTGGCTGGCG
GTTTGCCATAGCCGTTGCGACTGCTGCCAGTATTGCCAGCCATAGAGTCCACTTGCGCTT
AGCATGACCAAAATCAGCATCGCGACCAGCGTTTCAATCAGCGTATAACCACGTTGTGTT
TTCATGCCGGCAGTATGGAGCGAGGAGAAAAAAAGACGAGGGCCAGTTTCTATTTCTTCG
GCGCATCTTCCGGACTATTTACGCCGTTGCAGGACGTTGCAAAATTTCGGGAAGGCGTCT
CGAAGAATTTAACGGAGGGTAAAAAAACCGACGCACACTGGCGTCGGCTCTGGCAGGATG
TTTCGTAATTAGATAGCCACCGGCGCTTTattaaacctactATGACCATGATTACGGATT
CACTGGCCGTCGTTTTACAACGTCGTGACTGGGAAAACCCTGGCGTTACCCAACTTAATC
GCCTTGCAGCACATCCCCCTTTCGCCAGCTGGCGTAATAGCGAAGAGGCCCGCACCGATC
GCCCTTCCCAACAGTTGCGCAGCCTGAATGGCGAATGGCGCTTTGCCTGGTTTCCGGCAC
CAGAAGCGGTGCCGGAAAGCTGGCTGGAGTGCGATCTTCCTGAGGCCGATACTGTCGTCG
TCCCCTCAAACTGGCAGATGCACGGTTACGATGCGCCCATCTACACCAACGTGACCTATC
CCATTACGGTCAATCCGCCGTTTGTTCCCACGGAGAATCCGACGGGTTGTTACTCGCTCA
CATTTAATGTTGATGAAAGCTGGCTACAGGAAGGCCAGACGCGAATTATTTTTGATGGCG
TTAACTCGGCGTTTCATCTGTGGTGCAACGGGCGCTGGGTCGGTTACGGCCAGGACAGTC
GTTTGCCGTCTGAATTTGACCTGAGCGCATTTTTACGCGCCGGAGAAAACCGCCTCGCGG
TGATGGTGCTGCGCTGGAGTGACGGCAGTTATCTGGAAGATCAGGATATGTGGCGGATGA
GCGGCATTTTCCGTGACGTCTCGTTGCTGCATAAACCGACTACACAAATCAGCGATTTCC
ATGTTGCCACTCGCTTTAATGATGATTTCAGCCGCGCTGTACTGGAGGCTGAAGTTCAGA
TGTGCGGCGAGTTGCGTGACTACCTACGGGTAACAGTTTCTTTATGGCAGGGTGAAACGC
AGGTCGCCAGCGGCACCGCGCCTTTCGGCGGTGAAATTATCGATGAGCGTGGTGGTTATG
CCGATCGCGTCACACTACGTCTGAACGTCGAAAACCCGAAACTGTGGAGCGCCGAAATCC
CGAATCTCTATCGTGCGGTGGTTGAACTGCACACCGCCGACGGCACGCTGATTGAAGCAG
AAGCCTGCGATGTCGGTTTCCGCGAGGTGCGGATTGAAAATGGTCTGCTGCTGCTGAACG
GCAAGCCGTTGCTGATTCGAGGCGTTAACCGTCACGAGCATCATCCTCTGCATGGTCAGG
TCATGGATGAGCAGACGATGGTGCAGGATATCCTGCTGATGAAGCAGAACAACTTTAACG
CCGTGCGCTGTTCGCATTATCCGAACCATCCGCTGTGGTACACGCTGTGCGACCGCTACG
GCCTGTATGTGGTGGATGAAGCCAATATTGAAACCCACGGCATGGTGCCAATGAATCGTC
TGACCGATGATCCGCGCTGGCTACCGGCGATGAGCGAACGCGTAACGCGAATGGTGCAGC
GCGATCGTAATCACCCGAGTGTGATCATCTGGTCGCTGGGGAATGAATCAGGCCACGGCG
CTAATCACGACGCGCTGTATCGCTGGATCAAATCTGTCGATCCTTCCCGCCCGGTGCAGT
ATGAAGGCGGCGGAGCCGACACCACGGCCACCGATATTATTTGCCCGATGTACGCGCGCG
TGGATGAAGACCAGCCCTTCCCGGCTGTGCCGAAATGGTCCATCAAAAAATGGCTTTCGC
TACCTGGAGAGACGCGCCCGCTGATCCTTTGCGAATACGCCCACGCGATGGGTAACAGTC
TTGGCGGTTTCGCTAAATACTGGCAGGCGTTTCGTCAGTATCCCCGTTTACAGGGCGGCT
TCGTCTGGGACTGGGTGGATCAGTCGCTGATTAAATATGATGAAAACGGCAACCCGTGGT
CGGCTTACGGCGGTGATTTTGGCGATACGCCGAACGATCGCCAGTTCTGTATGAACGGTC
TGGTCTTTGCCGACCGCACGCCGCATCCAGCGCTGACGGAAGCAAAACACCAGCAGCAGT
TTTTCCAGTTCCGTTTATCCGGGCAAACCATCGAAGTGACCAGCGAATACCTGTTCCGTC
ATAGCGATAACGAGCTCCTGCACTGGATGGTGGCGCTGGATGGTAAGCCGCTGGCAAGCG
GTGAAGTGCCTCTGGATGTCGCTCCACAAGGTAAACAGTTGATTGAACTGCCTGAACTAC
CGCAGCCGGAGAGCGCCGGGCAACTCTGGCTCACAGTACGCGTAGTGCAACCGAACGCGA
CCGCATGGTCAGAAGCCGGGCACATCAGCGCCTGGCAGCAGTGGCGTCTGGCGGAAAACC
TCAGTGTGACGCTCCCCGCCGCGTCCCACGCCATCCCGCATCTGACCACCAGCGAAATGG
ATTTTTGCATCGAGCTGGGTAATAAGCGTTGGCAATTTAACCGCCAGTCAGGCTTTCTTT
CACAGATGTGGATTGGCGATAAAAAACAACTGtTGACGCCGCTGCGCGATCAGTTCACCC
GTGCACCGCTGGATAACGACATTGGCGTAAGTGAAGCGACCCGCATTGACCCTAACGCCT
GGGTCGAACGCTGGAAGGCGGCGGGCCATTACCAGGCCGAAGCAGCGTTGTTGCAGTGCA
CGGCAGATACACTTGCTGATGCGGTGCTGATTACGACCGCTCACGCGTGGCAGCATCAGG
GGAAAACCTTATTTATCAGCCGGAAAACCTACCGGATTGATGGTAGTGGTCAAATGGCGA
TTACCGTTGATGTTGAAGTGGCGAGCGATACACCGCATCCGGCGCGGATTGGCCTGAACT
GCCAGCTGGCGCAGGTAGCAGAGCGGGTAAACTGGCTCGGATTAGGGCCGCAAGAAAACT
ATCCCGACCGCCTTACTGCCGCCTGTTTTGACCGCTGGGATCTGCCATTGTCAGACATGT
ATACCCCGTACGTCTTCCCGAGCGAAAACGGTCTGCGCTGCGGGACGCGCGAATTGAATT
ATGGCCCACACCAGTGGCGCGGCGACTTCCAGTTCAACATCAGCCGCTACAGTCAACAGC
AACTGATGGAAACCAGCCATCGCCATCTGCTGCACGCGGAAGAAGGCACATGGCTGAATA
TCGACGGTTTCCATATGGGGATTGGTGGCGACGACTCCTGGAGCCCGTCAGTATCGGCGG
AATTCCAGCTGAGCGCCGGTCGCTACCATTACCAGTTGGTCTGGTGTCAAAAATAAGCGG
CCGCtTTATGTAGGCTGGAGCTGCTTCGAAGTTCCTATACTTTCTAGAGAATAGGAACTT
CGGAATAGGAACTTCAAGATCCCCTTATTAGAAGAACTCGTCAAGAAGGCGATAGAAGGC
GATGCGCTGCGAATCGGGAGCGGCGATACCGTAAAGCACGAGGAAGCGGTCAGCCCATTC
GCCGCCAAGCTCTTCAGCAATATCACGGGTAGCCAACGCTATGTCCTGATAGCGGTCCGC
CACACCCAGCCGGCCACAGTCGATGAATCCtGAAAAGCGGCCATTTTCCACCATGATATT
CGGCAAGCAGGCATCGCCATGGGTCACGACGAGATCCTCGCCGTCGGGCATGCGCGCCTT
GAGCCTGGCGAACAGTTCGGCTGGCGCGAGCCCCTGATGCTCTTCGTCCAGATCATCCTG
ATCGACAAGACCGGCTTCCATCCGAGTACGTGCTCGCTCGATGCGATGTTTCGCTTGGTG
GTCGAATGGGCAGGTAGCCGGATCAAGCGTATGCAGCCGCCGCATTGCATCAGCCATGAT
GGATACTTTCTCGGCAGGAGCAAGGTGAGATGACAGGAGATCCTGCCCCGGCACTTCGCC
CAATAGCAGCCAGTCCCTTCCCGCTTCAGTGACAACGTCGAGCACAGCTGCGCAAGGAAC
GCCCGTCGTGGCCAGCCACGATAGCCGCGCTGCCTCGTCCTGCAGTTCATTCAGGGCACC
GGACAGGTCGGTCTTGACAAAAAGAACCGGGCGCCCCTGCGCTGACAGCCGGAACACGGC
GGCATCAGAGCAGCCGATTGTCTGTTGTGCCCAGTCATAGCCGAATAGCCTCTCCACCCA
AGCGGCCGGAGAACCTGCGTGCAATCCATCTTGTTCAATCATGCGAAACGATCCTCATCC
TGTCTCTTGATCAGATCTTGATCCCCTGCGCCATCAGATCCTTGGCGGCAAGAAAGCCAT
CCAGTTTACTTTGCAGGGCTTCCCAACCTTACCAGAGGGCGCCCCAGCTGGCAATTCCGG
TTCGCTTGCTGTCCATAAAACCGCCCAGTCTAGCTATCGCCATGTAAGCCCACTGCAAGC
TACCTGCTTTCTCTTTGCGCTTGCGTTTTCCCTTGTCCAGATAGCCCAGTAGCTGACATT
CATCCGGGGTCAGCACCGTTTCTGCGGACTGGCTTTCTACGTGTTCCGCTTCCTTTAGCA
GCCCTTGCGCCCTGAGTGCTTGCGGCAGCGTGAGCTTCAAAAGCGCTCTGAAGTTCCTAT
ACTTTCTAGAGAATAGGAACTTCGAACTGCAGGTCGACGGATCCCCGGAATCATGGTTCC
TCAGGAAACGTGTTGCTGTGGGCTGCGACGATATGCCCAGACCATCATGATCACACCCGC
GACAATCATCGGGATGGAAAGAATTTGCCCCATGCTGATGTACTGCACCCAGGCACCGGT
AAACTGCGCGTCGGGCTGGCGGAAAAACTCAACAATGATGCGAAACGCGCCGTAACCAAT
CAGGAACAAACCTGAGACAGCTCCCATTGGGCGTGGTTTACGAATATACAGGTTGAGGAT
AATAAACAGCACCACACCTTCCAGCAGCAGCTCGTAAAGCTGTGATGGGTGGCGCGGCAG
CACACCGTAAGTGTCGAAAATGGATTGCCACTGCGGGTTGGTTTGCAGCAGCAAAATATC
TTCTGTACGGGAGCCAGGGAACAGCATGGCAAACGGGAAGTTCGGGTCAACGCGGCCCCA
CAATTCACCGTTAATAAAGTTGCCCAGACGCCCGGCACCAAGACCAAACGGAATGAGTGG
TGCGATAAAATCAGAGACCTGGAAGAAGGAACGTTTAGTACGGCGGGCGAAGATAATCAT
CACCACGATAACGCCAATCAGGCCGCCGTGGAAAGACATGCCGCCGTCCCAGACACGGAA
CAGATACAGCGGATCGGCCATAAACTGCGGGAAATTGTAGAACAGAACATAACCAATACG
TCCCCCGAGGAAGACGCCGAGGAAGCCCGCATAGAGTAAGTTTTCAACTTCATTTTTGGT
CCAGCCGCTGCCCGGACGATTCGCCCGTCGTGTTGCCAGCCACATTGCAAAAATGAAACC
CACCAGATACATCAGGCCGTACCAGTGAAGCGCCACGGGTCCTATTGAGAAAATGACCGG
ATCAAACTCCGGAAAATGCAGATAGCTACTGGTCATCTGTCACCACAAGTTCTTGTTATT
TCGCTGAAAGAGAACAGCGATTGAAATGCGCGCCGCAGGTTTCAGGCGCTCCAAAGGTGC
GAATAATAGCACAAGGGGACCTGGCTGGTTGCCGGATACCGTTAAAAGATATGTATA
pG292
>pG292, complete sequence.
SEQ ID NO: 5
TCGCGCGTTTCGGTGATGACGGTGAAAACCTCTGACACATGCAGCTCCCGGAGACGGTCA
CAGCTTGTCTGTAAGCGGATGCCGGGAGCAGACAAGCCCGTCAGGGCGCGTCAGCGGGTG
TTGGCGGGTGTCGGGGCTGGCTTAACTATGCGGCATCAGAGCAGATTGTACTGAGAGTGC
ACCATATATGCGGTGTGAAATACCGCACAGATGCGTAAGGAGAAAATACCGCATCAggcg
ccTCCTCAACCTGTATATTCGTAAACCACGCCCAATGGGAGCTGTCTCAGGTTTGTTCCT
GATTGGTTACGGCGCGTTTCGCATCATTGTTGAGTTTTTCCGCCAGCCCGACGCGCAGTT
TACCGGTGCCTGGGTGCAGTACATCAGCATGGGGCAAATTCTTTCCATCCCGATGATTGT
CGCGGGTGTGATCATGATGGTCTGGGCATATCGTCGCAGCCCACAGCAACACGTTTCCTG
AGGAACCATGAAACAGTATTTAGAACTGATGCAAAAAGTGCTCGACGAAGGCACACAGAA
AAACGACCGTACCGGAACCGGAACGCTTTCCATTTTTGGTCATCAGATGCGTTTTAACCT
GCAAGATGGATTCCCGCTGGTGACAACTAAACGTTGCCACCTGCGTTCCATCATCCATGA
ACTGCTGTGGTTTCTGCAGGGCGACACTAACATTGCTTATCTACACGAAAACAATGTCAC
CATCTGGGACGAATGGGCCGATGAAAACGGCGACCTCGGGCCAGTGTATGGTAAACAGTG
GCGCGCCTGGCCAACGCCAGATGGTCGTCATATTGACCAGATCACTACGGTACTGAACCA
GCTGAAAAACGACCCGGATTCGCGCCGCATTATTGTTTCAGCGTGGAACGTAGGCGAACT
GGATAAAATGGCGCTGGCACCGTGCCATGCATTCTTCCAGTTCTATGTGGCAGACGGCAA
ACTCTCTTGCCAGCTTTATCAGCGCTCCTGTGACGTCTTCCTCGGCCTGCCGTTCAACAT
TGCCAGCTACGCGTTATTGGTGCATATGATGGCGCAGCAGTGCGATCTGGAAGTGGGTGA
TTTTGTCTGGACCGGTGGCGACACGCATCTGTACAGCAACCATATGGATCAAACTCATCT
GCAATTAAGCCGCGAACCGCGTCCGCTGCCGAAGTTGATTATCAAACGTAAACCCGAATC
CATCTTCGACTACCGTTTCGAAGACTTTGAGATTGAAGGCTACGATCCGCATCCGGGCAT
TAAAGCGCCGGTGGCTATCTAATTACGAAACATCCTGCCAGAGCCGACGCCAGTGTGCGT
CGGTTTTTTTACCCTCCGTTAAATTCTTCGAGACGCCTTCCCGAAggcgccATTCGCCAT
TCAGGCTGCGCAACTGTTGGGAAGGGCGATCGGTGCGGGCCTCTTCGCTATTACGCCAGC
TGGCGAAAGGGGGATGTGCTGCAAGGCGATTAAGTTGGGTAACGCCAGGGTTTTCCCAGT
CACGACGTTGTAAAACGACGGCCAGTGCCAAGCTTACTGCTCACAAGAAAAAAGGCACGT
CATCTGACGTGCCTTTTTTATTTGTACTACCCTGTACGATTACTGCAGGTCGACTCTAGA
TGCATGCTCGAGTCAACGGTTTTTCAGCAATCGGTGCAAAATGCCGAAGTATTGCCTCAA
GGTAAACAGCCGCCGCATCCTGCCGTCTGCCGCAAAATCCAGCCACGCGCCGGCGGGCAG
CGTGTCCGTCCGTTTGAAGCATTGGTACAAAAACCGGCGGGCGCGTTCAAAATCTTCTTC
CGGCAAATGTTTCTCCAGCAATTCATACGCTACTGCTTTTATTTGGCGGTATTCAAGGCT
GTCGAACCGGGTTTTAAAACCCATAGACTGCAAAAAATCGTTTCTGGCGGTTTTTTGGAT
GCCTTGCGCGATTTCGTGTTGGCGGATGCTGTATTTGGATGAAACCTGATTGGCGTGAAG
GCGGTATTTGACCAAGGCTTCGGGATAATAAGCCAGCCTGCCCAATTTGCTGACATCGTA
CCAAAATTGGTAATCTTCCGCCCAATCCCGCTCGGTGTTGTAACGCAAACCGCCGTCAAT
GACGCTGCGCCTCATAATCATCGTGTTGTTGTGTATGGGGTTGCCGAAAGGGAAAAAGTC
GGCAATGTCTTCGTGTCGGGTCGGTTTTTTCCAAATTTTGCCGTGTTCGTGGTGCCGCGC
CAGCCGGTTGCCGTCCTTTTCTTCCGACAAAACTTCCAGCCACGCACCCATCGCGATGAT
GCTGCGGTCTTTTTCCATCTCACCCACGATTTTCTCAATCCAGTCGGGGGGGGCAATATC
GTCTGCATCGGTGCGCGCAATATATTCCCCCCCCCCCCCCGACTTTGCCAATTCATCCAG
CCCGATGTTTAAAGAGGGAATCAGACCGGAATTGCGCGGCTGCGCGAGGATGCGGATGCG
GCCGTCCTGTTCTTGGAAACGCTGGGCAATGGCAAGCGTACCGTCCGTCGAGCCGTCATC
GACAATCAAAATATCCAAGTTGCGCCAAGTTTGATTCACGACGGCGGCTAATGATTGGGC
GAAATATTTTTCTACGTTGTAGGCGCAAATCAATACGCTGACTAAAGGCTGCAATTTATT
CTCCCGATAGGCACGATGCCGTCTGAAGGCTTCAGACGGCATATGtatatctccttcttg
aaTTCTAACAATTGATTGAATGTATGCAAATAAATGCATACACCATAGGTGTGGTTTAAT
TTGATGCCCTTTTTCAGGGCTGGAATGTGTAAGAGCGGGGTTATTTATGCTGTTGTTTTT
TTGTTACTCGGGAAGGGCTTTACCTCTTCCGCATAAACGCTTCCATCAGCGTTTATAGTT
AAAAAAATCTTTCGGAACTGGTTTTGCGCTTACCCCAACCAACAGGGGATTTGCTGCTTT
CCATTGAGCCTGTTTCTCTGCGCGACGTTCGCGGCGGCGTGTTTGTGCATCCATCTGGAT
TCTCCTGTCAGTTAGCTTTGGTGGTGTGTGGCAGTTGTAGTCCTGAACGAAAACCCCCCG
CGATTGGCACATTGGCAGCTAATCCGGAATCGCACTTACGGCCAATGCTTCGTTTCGTAT
CACACACCCCAAAGCCTTCTGCTTTGAATGCTGCCCTTCTTCAGGGCTTAATTTTTAAGA
GCGTCACCTTCATGGTGGTCAGTGCGTCCTGCTGATGTGCTCAGTATCACCGCCAGTGGT
ATTTATGTCAACACCGCCAGAGATAATTTATCACCGCAGATGGTTATCTGTATGTTTTTT
ATATGAATTTATTTTTTGCAGGGGGGCATTGTTTGGTAGGTGAGAGATCAATTCTGCATT
AATGAATCGGCCAACGCGCGGGGAGAGGCGGTTTGCGTATTGGGCGCTCTTCCGCTTCCT
CGCTCACTGACTCGCTGCGCTCGGTCGTTCGGCTGCGGCGAGCGGTATCAGCTCACTCAA
AGGCGGTAATACGGTTATCCACAGAATCAGGGGATAACGCAGGAAAGAACATGTGAGCAA
AAGGCCAGCAAAAGGCCAGGAACCGTAAAAAGGCCGCGTTGCTGGCGTTTTTCCATAGGC
TCCGCCCCCCTGACGAGCATCACAAAAATCGACGCTCAAGTCAGAGGTGGCGAAACCCGA
CAGGACTATAAAGATACCAGGCGTTTCCCCCTGGAAGCTCCCTCGTGCGCTCTCCTGTTC
CGACCCTGCCGCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCCAAGCTGGGCT
GTGTGCACGAACCCCCCGTTCAGCCCGACCGCTGCGCCTTATCCGGTAACTATCGTCTTG
AGTCCAACCCGGTAAGACACGACTTATCGCCACTGGCAGCAGCCACTGGTAACAGGATTA
GCAGAGCGAGGTATGTAGGCGGTGCTACAGAGTTCTTGAAGTGGTGGCCTAACTACGGCT
ACACTAGAAGGACAGTATTTGGTATCTGCGCTCTGCTGAAGCCAGTTACCTTCGGAAAAA
GAGTTGGTAGCTCTTGATCCGGCAAACAAACCACCGCTGGTAGCGGTGGTTTTTTTGTTT
GCAAGCAGCAGATTACGCGCAGAAAAAAAGGATCTCAAGAAGATCCTTTGATCTTTTCTA
CGGGGTCTGACGCTCAGTGGAACGAAAACTCACGTTAAGGGATTTTGGTCATGAGATTAT
CAAAAAGGATCTTCACCTAGATCCTTTTAAATTAAAAATGAAGTTTTAAATCAATCTAAA
GTATATATGAGTAAACTTGGTCTGACAGTTACCAATGCTTAATCAGTGAGGCACCTATCT
CAGCGATCTGTCTATTTCGTTCATCCATAGTTGCCTGACTCCCCGTCGTGTAGATAACTA
CGATACGGGAGGGCTTACCATCTGGCCCCAGTGCTGCAATGATACCGCGAGACCCACGCT
CACCGGCTCCAGATTTATCAGCAATAAACCAGCCAGCCGGAAGGGCCGAGCGCAGAAGTG
GTCCTGCAACTTTATCCGCCTCCATCCAGTCTATTAATTGTTGCCGGGAAGCTAGAGTAA
GTAGTTCGCCAGTTAATAGTTTGCGCAACGTTGTTGCCATTGCTACAGGCATCGTGGTGT
CACGCTCGTCGTTTGGTATGGCTTCATTCAGCTCCGGTTCCCAACGATCAAGGCGAGTTA
CATGATCCCCCATGTTGTGCAAAAAAGCGGTTAGCTCCTTCGGTCCTCCGATCGTTGTCA
GAAGTAAGTTGGCCGCAGTGTTATCACTCATGGTTATGGCAGCACTGCATAATTCTCTTA
CTGTCATGCCATCCGTAAGATGCTTTTCTGTGACTGGTGAGTACTCAACCAAGTCATTCT
GAGAATAGTGTATGCGGCGACCGAGTTGCTCTTGCCCGGCGTCAATACGGGATAATACCG
CGCCACATAGCAGAACTTTAAAAGTGCTCATCATTGGAAAACGTTCTTCGGGGCGAAAAC
TCTCAAGGATCTTACCGCTGTTGAGATCCAGTTCGATGTAACCCACTCGTGCACCCAACT
GATCTTCAGCATCTTTTACTTTCACCAGCGTTTCTGGGTGAGCAAAAACAGGAAGGCAAA
ATGCCGCAAAAAAGGGAATAAGGGCGACACGGAAATGTTGAATACTCATACTCTTCCTTT
TTCAATATTATTGAAGCATTTATCAGGGTTATTGTCTCATGAGCGGATACATATTTGAAT
GTATTTAGAAAAATAAACAAATAGGGGTTCCGCGCACATTTCCCCGAAAAGTGCCACCTG
ACGTCTAAGAAACCATTATTATCATGACATTAACCTATAAAAATAGGCGTATCACGAGGC
CCTTTCGTC
pG221
>pG221, complete sequence.
SEQ ID NO: 6
TCGCGCGTTTCGGTGATGACGGTGAAAACCTCTGACACATGCAGCTCCCGGAGACGGTCA
CAGCTTGTCTGTAAGCGGATGCCGGGAGCAGACAAGCCCGTCAGGGCGCGTCAGCGGGTG
TTGGCGGGTGTCGGGGCTGGCTTAACTATGCGGCATCAGAGCAGATTGTACTGAGAGTGC
ACCATATATGCGGTGTGAAATACCGCACAGATGCGTAAGGAGAAAATACCGCATCAggcg
ccTCCTCAACCTGTATATTCGTAAACCACGCCCAATGGGAGCTGTCTCAGGTTTGTTCCT
GATTGGTTACGGCGCGTTTCGCATCATTGTTGAGTTTTTCCGCCAGCCCGACGCGCAGTT
TACCGGTGCCTGGGTGCAGTACATCAGCATGGGGCAAATTCTTTCCATCCCGATGATTGT
CGCGGGTGTGATCATGATGGTCTGGGCATATCGTCGCAGCCCACAGCAACACGTTTCCTG
AGGAACCATGAAACAGTATTTAGAACTGATGCAAAAAGTGCTCGACGAAGGCACACAGAA
AAACGACCGTACCGGAACCGGAACGCTTTCCATTTTTGGTCATCAGATGCGTTTTAACCT
GCAAGATGGATTCCCGCTGGTGACAACTAAACGTTGCCACCTGCGTTCCATCATCCATGA
ACTGCTGTGGTTTCTGCAGGGCGACACTAACATTGCTTATCTACACGAAAACAATGTCAC
CATCTGGGACGAATGGGCCGATGAAAACGGCGACCTCGGGCCAGTGTATGGTAAACAGTG
GCGCGCCTGGCCAACGCCAGATGGTCGTCATATTGACCAGATCACTACGGTACTGAACCA
GCTGAAAAACGACCCGGATTCGCGCCGCATTATTGTTTCAGCGTGGAACGTAGGCGAACT
GGATAAAATGGCGCTGGCACCGTGCCATGCATTCTTCCAGTTCTATGTGGCAGACGGCAA
ACTCTCTTGCCAGCTTTATCAGCGCTCCTGTGACGTCTTCCTCGGCCTGCCGTTCAACAT
TGCCAGCTACGCGTTATTGGTGCATATGATGGCGCAGCAGTGCGATCTGGAAGTGGGTGA
TTTTGTCTGGACCGGTGGCGACACGCATCTGTACAGCAACCATATGGATCAAACTCATCT
GCAATTAAGCCGCGAACCGCGTCCGCTGCCGAAGTTGATTATCAAACGTAAACCCGAATC
CATCTTCGACTACCGTTTCGAAGACTTTGAGATTGAAGGCTACGATCCGCATCCGGGCAT
TAAAGCGCCGGTGGCTATCTAATTACGAAACATCCTGCCAGAGCCGACGCCAGTGTGCGT
CGGTTTTTTTACCCTCCGTTAAATTCTTCGAGACGCCTTCCCGAAggcgccATTCGCCAT
TCAGGCTGCGCAACTGTTGGGAAGGGCGATCGGTGCGGGCCTCTTCGCTATTACGCCAGC
TGGCGAAAGGGGGATGTGCTGCAAGGCGATTAAGTTGGGTAACGCCAGGGTTTTCCCAGT
CACGACGTTGTAAAACGACGGCCAGTGCCAAGCTTACTGCTCACAAGAAAAAAGGCACGT
CATCTGACGTGCCTTTTTTATTTGTACTACCCTGTACGATTACTGCAGGTCGACTCTAGA
TGCATGCTCGAGTTATTATTTAATATATTTACAATAGATGAAGGACGCAATCGTACGGAT
ACCGCCGAACAGGTAGTTAATGTTACCGGTCAGGAAGAAGCACTTCATTTTGATAACCAG
GTCGTTAACCATCACCATGTACAGGTTTTTTTTTGCGGTAGACTGACCTTCGTGCAGGCG
GTAGTAGAACAGGTATTCCGGCAGGTTTTGGAACTTGATTTTTGCCAGGCTCAGACGGTT
CCACAGCTCGTAATCTTCGGAGTAGTTAGAAAACATATAACCACCGATGCTCGCGATGAC
TTTTTTACGAAACATTACGCTCGGGTGAACAATACAACACTTATACGGCAGGTTTTTAAC
GATGTCCAGGTTCTCTTCCGGCAGTTTGGTCTTGTTGATTTCACGACCTTTGTCGTCAAT
AAAGATTGCGTTGGTACCCACAACATCTACGTACGGATTGTTCTTCAGGAAGTCAACCTG
TTTAGTAAAACGGTCCGGGTGAGAGATGTCGTCAGAGTCCATACGGGCAATAAATTCGCC
GTTGCTCAGGTCGATCGCTTTGTTCAGGGAGTACGGCAGGTAAGCGATGTTAGTGCGGAT
CAGTTTGATTTTGTCGTTAACTTTGTGTTTCAGTTCGTTATAGAAGTCGTCAGTGCAGCA
GTTCGCAACGATGATGATTTCGAAGCTGCTGAAGGTCTGAGACAGGATGCTGTTGATCGC
TTCGTCCAGAAAAGGGTTTTTCTTGTTAACAGGCAGGATAACGCTCACAACCGGGTGGGT
AGATTCCGCGGATTCCGCTTCATCGATGATCATATGTATATCTCCTTCTTCTCGAGTCAA
CGGTTTTTCAGCAATCGGTGCAAAATGCCGAAGTATTGCCTCAAGGTAAACAGCCGCCGC
ATCCTGCCGTCTGCCGCAAAATCCAGCCACGCGCCGGCGGGCAGCGTGTCCGTCCGTTTG
AAGCATTGGTACAAAAACCGGCGGGCGCGTTCAAAATCTTCTTCCGGCAAATGTTTCTCC
AGCAATTCATACGCTACTGCTTTTATTTGGCGGTATTCAAGGCTGTCGAACCGGGTTTTA
AAACCCATAGACTGCAAAAAATCGTTTCTGGCGGTTTTTTGGATGCCTTGCGCGATTTCG
TGTTGGCGGATGCTGTATTTGGATGAAACCTGATTGGCGTGAAGGCGGTATTTGACCAAG
GCTTCGGGATAATAAGCCAGCCTGCCCAATTTGCTGACATCGTACCAAAATTGGTAATCT
TCCGCCCAATCCCGCTCGGTGTTGTAACGCAAACCGCCGTCAATGACGCTGCGCCTCATA
ATCATCGTGTTGTTGTGTATGGGGTTGCCGAAAGGGAAAAAGTCGGCAATGTCTTCGTGT
CGGGTCGGTTTTTTCCAAATTTTGCCGTGTTCGTGGTGCCGCGCCAGCCGGTTGCCGTCC
TTTTCTTCCGACAAAACTTCCAGCCACGCACCCATCGCGATGATGCTGCGGTCTTTTTCC
ATCTCACCCACGATTTTCTCAATCCAGTCGGGGGCGGCAATATCGTCTGCATCGGTGCGC
GCAATATATTCCCCCCCCCCCCCCGACTTTGCCAATTCATCCAGCCCGATGTTTAAAGAG
GGAATCAGACCGGAATTGCGCGGCTGCGCGAGGATGCGGATGCGGCCGTCCTGTTCTTGG
AAACGCTGGGCAATGGCAAGCGTACCGTCCGTCGAGCCGTCATCGACAATCAAAATATCC
AAGTTGCGCCAAGTTTGATTCACGACGGCGGCTAATGATTGGGCGAAATATTTTTCTACG
TTGTAGGCGCAAATCAATACGCTGACTAAAGGCTGCAATTTATTCTCCCGATAGGCACGA
TGCCGTCTGAAGGCTTCAGACGGCATATGtatatctccttcttgaaTTCTAACAATTGAT
TGAATGTATGCAAATAAATGCATACACCATAGGTGTGGTTTAATTTGATGCCCTTTTTCA
GGGCTGGAATGTGTAAGAGCGGGGTTATTTATGCTGTTGTTTTTTTGTTACTCGGGAAGG
GCTTTACCTCTTCCGCATAAACGCTTCCATCAGCGTTTATAGTTAAAAAAATCTTTCGGA
ACTGGTTTTGCGCTTACCCCAACCAACAGGGGATTTGCTGCTTTCCATTGAGCCTGTTTC
TCTGCGCGACGTTCGCGGCGGCGTGTTTGTGCATCCATCTGGATTCTCCTGTCAGTTAGC
TTTGGTGGTGTGTGGCAGTTGTAGTCCTGAACGAAAACCCCCCGCGATTGGCACATTGGC
AGCTAATCCGGAATCGCACTTACGGCCAATGCTTCGTTTCGTATCACACACCCCAAAGCC
TTCTGCTTTGAATGCTGCCCTTCTTCAGGGCTTAATTTTTAAGAGCGTCACCTTCATGGT
GGTCAGTGCGTCCTGCTGATGTGCTCAGTATCACCGCCAGTGGTATTTATGTCAACACCG
CCAGAGATAATTTATCACCGCAGATGGTTATCTGTATGTTTTTTATATGAATTTATTTTT
TGCAGGGGGGCATTGTTTGGTAGGTGAGAGATCAATTCTGCATTAATGAATCGGCCAACG
CGCGGGGAGAGGCGGTTTGCGTATTGGGCGCTCTTCCGCTTCCTCGCTCACTGACTCGCT
GCGCTCGGTCGTTCGGCTGCGGCGAGCGGTATCAGCTCACTCAAAGGCGGTAATACGGTT
ATCCACAGAATCAGGGGATAACGCAGGAAAGAACATGTGAGCAAAAGGCCAGCAAAAGGC
CAGGAACCGTAAAAAGGCCGCGTTGCTGGCGTTTTTCCATAGGCTCCGCCCCCCTGACGA
GCATCACAAAAATCGACGCTCAAGTCAGAGGTGGCGAAACCCGACAGGACTATAAAGATA
CCAGGCGTTTCCCCCTGGAAGCTCCCTCGTGCGCTCTCCTGTTCCGACCCTGCCGCTTAC
CGGATACCTGTCCGCCTTTCTCCCTTCGGGAAGCGTGGCGCTTTCTCATAGCTCACGCTG
TAGGTATCTCAGTTCGGTGTAGGTCGTTCGCTCCAAGCTGGGCTGTGTGCACGAACCCCC
CGTTCAGCCCGACCGCTGCGCCTTATCCGGTAACTATCGTCTTGAGTCCAACCCGGTAAG
ACACGACTTATCGCCACTGGCAGCAGCCACTGGTAACAGGATTAGCAGAGCGAGGTATGT
AGGCGGTGCTACAGAGTTCTTGAAGTGGTGGCCTAACTACGGCTACACTAGAAGGACAGT
ATTTGGTATCTGCGCTCTGCTGAAGCCAGTTACCTTCGGAAAAAGAGTTGGTAGCTCTTG
ATCCGGCAAACAAACCACCGCTGGTAGCGGTGGTTTTTTTGTTTGCAAGCAGCAGATTAC
GCGCAGAAAAAAAGGATCTCAAGAAGATCCTTTGATCTTTTCTACGGGGTCTGACGCTCA
GTGGAACGAAAACTCACGTTAAGGGATTTTGGTCATGAGATTATCAAAAAGGATCTTCAC
CTAGATCCTTTTAAATTAAAAATGAAGTTTTAAATCAATCTAAAGTATATATGAGTAAAC
TTGGTCTGACAGTTACCAATGCTTAATCAGTGAGGCACCTATCTCAGCGATCTGTCTATT
TCGTTCATCCATAGTTGCCTGACTCCCCGTCGTGTAGATAACTACGATACGGGAGGGCTT
ACCATCTGGCCCCAGTGCTGCAATGATACCGCGAGACCCACGCTCACCGGCTCCAGATTT
ATCAGCAATAAACCAGCCAGCCGGAAGGGCCGAGCGCAGAAGTGGTCCTGCAACTTTATC
CGCCTCCATCCAGTCTATTAATTGTTGCCGGGAAGCTAGAGTAAGTAGTTCGCCAGTTAA
TAGTTTGCGCAACGTTGTTGCCATTGCTACAGGCATCGTGGTGTCACGCTCGTCGTTTGG
TATGGCTTCATTCAGCTCCGGTTCCCAACGATCAAGGCGAGTTACATGATCCCCCATGTT
GTGCAAAAAAGCGGTTAGCTCCTTCGGTCCTCCGATCGTTGTCAGAAGTAAGTTGGCCGC
AGTGTTATCACTCATGGTTATGGCAGCACTGCATAATTCTCTTACTGTCATGCCATCCGT
AAGATGCTTTTCTGTGACTGGTGAGTACTCAACCAAGTCATTCTGAGAATAGTGTATGCG
GCGACCGAGTTGCTCTTGCCCGGCGTCAATACGGGATAATACCGCGCCACATAGCAGAAC
TTTAAAAGTGCTCATCATTGGAAAACGTTCTTCGGGGCGAAAACTCTCAAGGATCTTACC
GCTGTTGAGATCCAGTTCGATGTAACCCACTCGTGCACCCAACTGATCTTCAGCATCTTT
TACTTTCACCAGCGTTTCTGGGTGAGCAAAAACAGGAAGGCAAAATGCCGCAAAAAAGGG
AATAAGGGCGACACGGAAATGTTGAATACTCATACTCTTCCTTTTTCAATATTATTGAAG
CATTTATCAGGGTTATTGTCTCATGAGCGGATACATATTTGAATGTATTTAGAAAAATAA
ACAAATAGGGGTTCCGCGCACATTTCCCCGAAAAGTGCCACCTGACGTCTAAGAAACCAT
TATTATCATGACATTAACCTATAAAAATAGGCGTATCACGAGGCCCTTTCGTC
pG293
>pG293, complete sequence.
SEQ ID NO: 7
TCGCGCGTTTCGGTGATGACGGTGAAAACCTCTGACACATGCAGCTCCCGGAGACGGTCA
CAGCTTGTCTGTAAGCGGATGCCGGGAGCAGACAAGCCCGTCAGGGCGCGTCAGCGGGTG
TTGGCGGGTGTCGGGGCTGGCTTAACTATGCGGCATCAGAGCAGATTGTACTGAGAGTGC
ACCATATATGCGGTGTGAAATACCGCACAGATGCGTAAGGAGAAAATACCGCATCAggcg
ccTCCTCAACCTGTATATTCGTAAACCACGCCCAATGGGAGCTGTCTCAGGTTTGTTCCT
GATTGGTTACGGCGCGTTTCGCATCATTGTTGAGTTTTTCCGCCAGCCCGACGCGCAGTT
TACCGGTGCCTGGGTGCAGTACATCAGCATGGGGCAAATTCTTTCCATCCCGATGATTGT
CGCGGGTGTGATCATGATGGTCTGGGCATATCGTCGCAGCCCACAGCAACACGTTTCCTG
AGGAACCATGAAACAGTATTTAGAACTGATGCAAAAAGTGCTCGACGAAGGCACACAGAA
AAACGACCGTACCGGAACCGGAACGCTTTCCATTTTTGGTCATCAGATGCGTTTTAACCT
GCAAGATGGATTCCCGCTGGTGACAACTAAACGTTGCCACCTGCGTTCCATCATCCATGA
ACTGCTGTGGTTTCTGCAGGGCGACACTAACATTGCTTATCTACACGAAAACAATGTCAC
CATCTGGGACGAATGGGCCGATGAAAACGGCGACCTCGGGCCAGTGTATGGTAAACAGTG
GCGCGCCTGGCCAACGCCAGATGGTCGTCATATTGACCAGATCACTACGGTACTGAACCA
GCTGAAAAACGACCCGGATTCGCGCCGCATTATTGTTTCAGCGTGGAACGTAGGCGAACT
GGATAAAATGGCGCTGGCACCGTGCCATGCATTCTTCCAGTTCTATGTGGCAGACGGCAA
ACTCTCTTGCCAGCTTTATCAGCGCTCCTGTGACGTCTTCCTCGGCCTGCCGTTCAACAT
TGCCAGCTACGCGTTATTGGTGCATATGATGGCGCAGCAGTGCGATCTGGAAGTGGGTGA
TTTTGTCTGGACCGGTGGCGACACGCATCTGTACAGCAACCATATGGATCAAACTCATCT
GCAATTAAGCCGCGAACCGCGTCCGCTGCCGAAGTTGATTATCAAACGTAAACCCGAATC
CATCTTCGACTACCGTTTCGAAGACTTTGAGATTGAAGGCTACGATCCGCATCCGGGCAT
TAAAGCGCCGGTGGCTATCTAATTACGAAACATCCTGCCAGAGCCGACGCCAGTGTGCGT
CGGTTTTTTTACCCTCCGTTAAATTCTTCGAGACGCCTTCCCGAAggcgccATTCGCCAT
TCAGGCTGCGCAACTGTTGGGAAGGGCGATCGGTGCGGGCCTCTTCGCTATTACGCCAGC
TGGCGAAAGGGGGATGTGCTGCAAGGCGATTAAGTTGGGTAACGCCAGGGTTTTCCCAGT
CACGACGTTGTAAAACGACGGCCAGTGCCAAGCTTACTGCTCACAAGAAAAAAGGCACGT
CATCTGACGTGCCTTTTTTATTTGTACTACCCTGTACGATTACTGCAGGTCGACTCTAGA
TGCATGCTCGAGTTACTATAAAAATCTCCTGATAACTTTTTTATATATATAGCCACGAAA
ACTAGTGGGAAGAAGTCTAACAAATATCCTTGTGATAATATTTATTATAAAGTCTTGATA
TGATATATAATTTGCACGATAAAAATGATACTGAGCTTTAATTTCTGAAATGGCATATTT
TATTCCACCTCGTCTTTTTGCTGTTTCCTTTGAAAATCTCATATCAACTAAAACTTGTTG
AATATTATCACCATTACATCCATTAGCTAAAGATTTCAACCAAAGGGCATAATCTTCATA
TAAGTACTCATTTCCATAACCGCCGACGCGCATTATTTCACACTTTTTAAATGCAACTGC
AGGGTGATTAAAAGGAGATCTGTTTTTTGAATATTTAAGTATAGAATTCCGACTTGGTAC
TTTTTTTATTATGCCCTTATCTCCTGGATTGAATTCGAACTCTTCAACCCAAGAGCTAAG
AATATGAACATCACCATTCTTAGTCATAAAATCAAGTATACATTCGAATCGATTTGTTCT
ATTTATATCATCAGAATCAGCACGTATTACTAAATCATGCATACATTGATTCAACCCAAA
ATTTAACGCTGCCCCCAACCCCATATTTTTTTCAAGTGTGAAATCTTTTATATTTAAATA
TCTTCTCCAACTATCAATAACAGAATTGAGTTCAGATGTGACCTTACCATCACGAATAAT
AATAACTTCATCTGGGGCAACCGTATTTTTATAAATTGATTGTAAAGCCTCAGAGAGAAA
TAGGGGAGAATCCTTGATGTATATAGCCATCAAAACAGAAAACTTTGGAGTGTCATCTGA
CATATGTATATCTCCTTCTTCTCGAGTCAACGGTTTTTCAGCAATCGGTGCAAAATGCCG
AAGTATTGCCTCAAGGTAAACAGCCGCCGCATCCTGCCGTCTGCCGCAAAATCCAGCCAC
GCGCCGGCGGGCAGCGTGTCCGTCCGTTTGAAGCATTGGTACAAAAACCGGCGGGCGCGT
TCAAAATCTTCTTCCGGCAAATGTTTCTCCAGCAATTCATACGCTACTGCTTTTATTTGG
CGGTATTCAAGGCTGTCGAACCGGGTTTTAAAACCCATAGACTGCAAAAAATCGTTTCTG
GCGGTTTTTTGGATGCCTTGCGCGATTTCGTGTTGGCGGATGCTGTATTTGGATGAAACC
TGATTGGCGTGAAGGCGGTATTTGACCAAGGCTTCGGGATAATAAGCCAGCCTGCCCAAT
TTGCTGACATCGTACCAAAATTGGTAATCTTCCGCCCAATCCCGCTCGGTGTTGTAACGC
AAACCGCCGTCAATGACGCTGCGCCTCATAATCATCGTGTTGTTGTGTATGGGGTTGCCG
AAAGGGAAAAAGTCGGCAATGTCTTCGTGTCGGGTCGGTTTTTTCCAAATTTTGCCGTGT
TCGTGGTGCCGCGCCAGCCGGTTGCCGTCCTTTTCTTCCGACAAAACTTCCAGCCACGCA
CCCATCGCGATGATGCTGCGGTCTTTTTCCATCTCACCCACGATTTTCTCAATCCAGTCG
GGGGCGGCAATATCGTCTGCATCGGTGCGCGCAATATATTCCCCCCCCCCCCCCGACTTT
GCCAATTCATCCAGCCCGATGTTTAAAGAGGGAATCAGACCGGAATTGCGCGGCTGCGCG
AGGATGCGGATGCGGCCGTCCTGTTCTTGGAAACGCTGGGCAATGGCAAGCGTACCGTCC
GTCGAGCCGTCATCGACAATCAAAATATCCAAGTTGCGCCAAGTTTGATTCACGACGGCG
GCTAATGATTGGGCGAAATATTTTTCTACGTTGTAGGCGCAAATCAATACGCTGACTAAA
GGCTGCAATTTATTCTCCCGATAGGCACGATGCCGTCTGAAGGCTTCAGACGGCATATGt
atatctccttcttgaaTTCTAACAATTGATTGAATGTATGCAAATAAATGCATACACCAT
AGGTGTGGTTTAATTTGATGCCCTTTTTCAGGGCTGGAATGTGTAAGAGCGGGGTTATTT
ATGCTGTTGTTTTTTTGTTACTCGGGAAGGGCTTTACCTCTTCCGCATAAACGCTTCCAT
CAGCGTTTATAGTTAAAAAAATCTTTCGGAACTGGTTTTGCGCTTACCCCAACCAACAGG
GGATTTGCTGCTTTCCATTGAGCCTGTTTCTCTGCGCGACGTTCGCGGCGGCGTGTTTGT
GCATCCATCTGGATTCTCCTGTCAGTTAGCTTTGGTGGTGTGTGGCAGTTGTAGTCCTGA
ACGAAAACCCCCCGCGATTGGCACATTGGCAGCTAATCCGGAATCGCACTTACGGCCAAT
GCTTCGTTTCGTATCACACACCCCAAAGCCTTCTGCTTTGAATGCTGCCCTTCTTCAGGG
CTTAATTTTTAAGAGCGTCACCTTCATGGTGGTCAGTGCGTCCTGCTGATGTGCTCAGTA
TCACCGCCAGTGGTATTTATGTCAACACCGCCAGAGATAATTTATCACCGCAGATGGTTA
TCTGTATGTTTTTTATATGAATTTATTTTTTGCAGGGGGGCATTGTTTGGTAGGTGAGAG
ATCAATTCTGCATTAATGAATCGGCCAACGCGCGGGGAGAGGCGGTTTGCGTATTGGGCG
CTCTTCCGCTTCCTCGCTCACTGACTCGCTGCGCTCGGTCGTTCGGCTGCGGCGAGCGGT
ATCAGCTCACTCAAAGGCGGTAATACGGTTATCCACAGAATCAGGGGATAACGCAGGAAA
GAACATGTGAGCAAAAGGCCAGCAAAAGGCCAGGAACCGTAAAAAGGCCGCGTTGCTGGC
GTTTTTCCATAGGCTCCGCCCCCCTGACGAGCATCACAAAAATCGACGCTCAAGTCAGAG
GTGGCGAAACCCGACAGGACTATAAAGATACCAGGCGTTTCCCCCTGGAAGCTCCCTCGT
GCGCTCTCCTGTTCCGACCCTGCCGCTTACCGGATACCTGTCCGCCTTTCTCCCTTCGGG
AAGCGTGGCGCTTTCTCATAGCTCACGCTGTAGGTATCTCAGTTCGGTGTAGGTCGTTCG
CTCCAAGCTGGGCTGTGTGCACGAACCCCCCGTTCAGCCCGACCGCTGCGCCTTATCCGG
TAACTATCGTCTTGAGTCCAACCCGGTAAGACACGACTTATCGCCACTGGCAGCAGCCAC
TGGTAACAGGATTAGCAGAGCGAGGTATGTAGGCGGTGCTACAGAGTTCTTGAAGTGGTG
GCCTAACTACGGCTACACTAGAAGaACAGTATTTGGTATCTGCGCTCTGCTGAAGCCAGT
TACCTTCGGAAAAAGAGTTGGTAGCTCTTGATCCGGCAAACAAACCACCGCTGGTAGCGG
TGGTTTTTTTGTTTGCAAGCAGCAGATTACGCGCAGAAAAAAAGGATCTCAAGAAGATCC
TTTGATCTTTTCTACGGGGTCTGACGCTCAGTGGAACGAAAACTCACGTTAAGGGATTTT
GGTCATGAGATTATCAAAAAGGATCTTCACCTAGATCCTTTTAAATTAAAAATGAAGTTT
TAAATCAATCTAAAGTATATATGAGTAAACTTGGTCTGACAGTTACCAATGCTTAATCAG
TGAGGCACCTATCTCAGCGATCTGTCTATTTCGTTCATCCATAGTTGCCTGACTCCCCGT
CGTGTAGATAACTACGATACGGGAGGGCTTACCATCTGGCCCCAGTGCTGCAATGATACC
GCGAGACCCACGCTCACCGGCTCCAGATTTATCAGCAATAAACCAGCCAGCCGGAAGGGC
CGAGCGCAGAAGTGGTCCTGCAACTTTATCCGCCTCCATCCAGTCTATTAATTGTTGCCG
GGAAGCTAGAGTAAGTAGTTCGCCAGTTAATAGTTTGCGCAACGTTGTTGCCATTGCTAC
AGGCATCGTGGTGTCACGCTCGTCGTTTGGTATGGCTTCATTCAGCTCCGGTTCCCAACG
ATCAAGGCGAGTTACATGATCCCCCATGTTGTGCAAAAAAGCGGTTAGCTCCTTCGGTCC
TCCGATCGTTGTCAGAAGTAAGTTGGCCGCAGTGTTATCACTCATGGTTATGGCAGCACT
GCATAATTCTCTTACTGTCATGCCATCCGTAAGATGCTTTTCTGTGACTGGTGAGTACTC
AACCAAGTCATTCTGAGAATAGTGTATGCGGCGACCGAGTTGCTCTTGCCCGGCGTCAAT
ACGGGATAATACCGCGCCACATAGCAGAACTTTAAAAGTGCTCATCATTGGAAAACGTTC
TTCGGGGCGAAAACTCTCAAGGATCTTACCGCTGTTGAGATCCAGTTCGATGTAACCCAC
TCGTGCACCCAACTGATCTTCAGCATCTTTTACTTTCACCAGCGTTTCTGGGTGAGCAAA
AACAGGAAGGCAAAATGCCGCAAAAAAGGGAATAAGGGCGACACGGAAATGTTGAATACT
CATACTCTTCCTTTTTCAATATTATTGAAGCATTTATCAGGGTTATTGTCTCATGAGCGG
ATACATATTTGAATGTATTTAGAAAAATAAACAAATAGGGGTTCCGCGCACATTTCCCCG
AAAAGTGCCACCTGACGTCTAAGAAACCATTATTATCATGACATTAACCTATAAAAATAG
GCGTATCACGAGGCCCTTTCGTC
pG294
>pG294, complete sequence.
SEQ ID NO: 8
TCGCGCGTTTCGGTGATGACGGTGAAAACCTCTGACACATGCAGCTCCCGGAGACGGTCA
CAGCTTGTCTGTAAGCGGATGCCGGGAGCAGACAAGCCCGTCAGGGCGCGTCAGCGGGTG
TTGGCGGGTGTCGGGGCTGGCTTAACTATGCGGCATCAGAGCAGATTGTACTGAGAGTGC
ACCATATATGCGGTGTGAAATACCGCACAGATGCGTAAGGAGAAAATACCGCATCAggcg
CCTCCTCAACCTGTATATTCGTAAACCACGCCCAATGGGAGCTGTCTCAGGTTTGTTCCT
GATTGGTTACGGCGCGTTTCGCATCATTGTTGAGTTTTTCCGCCAGCCCGACGCGCAGTT
TACCGGTGCCTGGGTGCAGTACATCAGCATGGGGCAAATTCTTTCCATCCCGATGATTGT
CGCGGGTGTGATCATGATGGTCTGGGCATATCGTCGCAGCCCACAGCAACACGTTTCCTG
AGGAACCATGAAACAGTATTTAGAACTGATGCAAAAAGTGCTCGACGAAGGCACACAGAA
AAACGACCGTACCGGAACCGGAACGCTTTCCATTTTTGGTCATCAGATGCGTTTTAACCT
GCAAGATGGATTCCCGCTGGTGACAACTAAACGTTGCCACCTGCGTTCCATCATCCATGA
ACTGCTGTGGTTTCTGCAGGGCGACACTAACATTGCTTATCTACACGAAAACAATGTCAC
CATCTGGGACGAATGGGCCGATGAAAACGGCGACCTCGGGCCAGTGTATGGTAAACAGTG
GCGCGCCTGGCCAACGCCAGATGGTCGTCATATTGACCAGATCACTACGGTACTGAACCA
GCTGAAAAACGACCCGGATTCGCGCCGCATTATTGTTTCAGCGTGGAACGTAGGCGAACT
GGATAAAATGGCGCTGGCACCGTGCCATGCATTCTTCCAGTTCTATGTGGCAGACGGCAA
ACTCTCTTGCCAGCTTTATCAGCGCTCCTGTGACGTCTTCCTCGGCCTGCCGTTCAACAT
TGCCAGCTACGCGTTATTGGTGCATATGATGGCGCAGCAGTGCGATCTGGAAGTGGGTGA
TTTTGTCTGGACCGGTGGCGACACGCATCTGTACAGCAACCATATGGATCAAACTCATCT
GCAATTAAGCCGCGAACCGCGTCCGCTGCCGAAGTTGATTATCAAACGTAAACCCGAATC
CATCTTCGACTACCGTTTCGAAGACTTTGAGATTGAAGGCTACGATCCGCATCCGGGCAT
TAAAGCGCCGGTGGCTATCTAATTACGAAACATCCTGCCAGAGCCGACGCCAGTGTGCGT
CGGTTTTTTTACCCTCCGTTAAATTCTTCGAGACGCCTTCCCGAAggcgccATTCGCCAT
TCAGGCTGCGCAACTGTTGGGAAGGGCGATCGGTGCGGGCCTCTTCGCTATTACGCCAGC
TGGCGAAAGGGGGATGTGCTGCAAGGCGATTAAGTTGGGTAACGCCAGGGTTTTCCCAGT
CACGACGTTGTAAAACGACGGCCAGTGCCAAGCTTACTGCTCACAAGAAAAAAGGCACGT
CATCTGACGTGCCTTTTTTATTTGTACTACCCTGTACGATTACTGCAGGTCGACTCTAGA
TGCATGCTCGAGTTATTAGATTTCCTTCGGCGTCTTCAGCGGGGCATACTGCTTAGAATA
CTTATGGAATGCACGCAGACATTTCTTGGTATTCAGCTGGTCGTAGAATAAGTTGTCGAA
GTAGCTCTTCTTCGGGCTTTTCAGGCTGATATCTGGTTTCTTAGCGTTCTGGATCGTAGA
GTTAAACGCGTGTTTATTCAGGCTTACCGGACACGGTAGGTAAGTGAAGTTCGCAACGTC
GTGATAGGTCGGATTATTCATAAACATGTCAACAGGTTCGATAATCTTGAATCTTTCGGT
CGCCTCGATGAAAGTCTTTGCACCTTTCGGAGTCAAATAATAACCAGCGGTCGTTGCAAC
GTAGACAGAAGTCAAATAGAAGTGTTCCAGGAACAAAGCCTCCTTCTCATTGTCCGCTCT
GCGCTCCCAGAAGGTTTCCTTAATATTGGTTTCCTTCGCTAGCAGACTCTCATACTTACC
CGTtAACTCGTCATACTTCTTcGTtAAaTCGTCATACTTCTTaGTtAAATCGTCATACTT
CGCAACCACGTTCTTGTAGACTTCGGTACGCGCCAGGATGGAGCCGATAAAACGGTTCAG
TTTGCGTTTGATTTTTTTGAAAACTACCTGGTTGAAGCTAATTTTCTGCGGCGCGATCTT
ACACGGGTCGCTCGGTTCCTTCGGATCCTGCTGAGTCTCGATGATGAAGTCCTGCTGGGT
GTCCCTGGCCGGGTTCGGCGGCGGTGGAGTTACCTCGTGATTCTCGATCGGTACCTCCGC
TTCAGTGTAGATCGGCAACGCACATAGGTTCGTCTTGTGACCACCCCAGTAGTGGCCATA
CAGGCGAACGAAGTCGAACGGAGACTTTAGACAGTCCTCCAGAGCCTGCATAAAGTTGCT
TTCCAGAGCGACGTCGTCCTCCAGGATGACAACTGGCTGATTAGTCTTTACACACTCCTT
CCATAGGAAGTAGTGACCCAGGTAACAACCGAACTCACGCGGTAGCAGCTCCTGGTAGCA
GTAGTCGCTGTGAAACCAGTCACTCTTCAGCATGGACTGGGCGTCGTACAATTCCTGGAC
GAACTTCTCGAAGTCCTCATGCTTCGGAGAGATCGCATCGAAAATCTGGAATACACATCT
ACCCTTGAATTTCTCGTTACTCTCACTGACCAACTTCTCGGTGTCTAGCCTACGCTGGGA
CTCCTTCAGGCTGATAATGTATACGCCGATCATATGTATATCTCCTTCTTCTCGAGTCAA
CGGTTTTTCAGCAATCGGTGCAAAATGCCGAAGTATTGCCTCAAGGTAAACAGCCGCCGC
ATCCTGCCGTCTGCCGCAAAATCCAGCCACGCGCCGGCGGGCAGCGTGTCCGTCCGTTTG
AAGCATTGGTACAAAAACCGGCGGGCGCGTTCAAAATCTTCTTCCGGCAAATGTTTCTCC
AGCAATTCATACGCTACTGCTTTTATTTGGCGGTATTCAAGGCTGTCGAACCGGGTTTTA
AAACCCATAGACTGCAAAAAATCGTTTCTGGCGGTTTTTTGGATGCCTTGCGCGATTTCG
TGTTGGCGGATGCTGTATTTGGATGAAACCTGATTGGCGTGAAGGCGGTATTTGACCAAG
GCTTCGGGATAATAAGCCAGCCTGCCCAATTTGCTGACATCGTACCAAAATTGGTAATCT
TCCGCCCAATCCCGCTCGGTGTTGTAACGCAAACCGCCGTCAATGACGCTGCGCCTCATA
ATCATCGTGTTGTTGTGTATGGGGTTGCCGAAAGGGAAAAAGTCGGCAATGTCTTCGTGT
CGGGTCGGTTTTTTCCAAATTTTGCCGTGTTCGTGGTGCCGCGCCAGCCGGTTGCCGTCC
TTTTCTTCCGACAAAACTTCCAGCCACGCACCCATCGCGATGATGCTGCGGTCTTTTTCC
ATCTCACCCACGATTTTCTCAATCCAGTCGGGGGCGGCAATATCGTCTGCATCGGTGCGC
GCAATATATTCCCCCCCCCCCCCCGACTTTGCCAATTCATCCAGCCCGATGTTTAAAGAG
GGAATCAGACCGGAATTGCGCGGCTGCGCGAGGATGCGGATGCGGCCGTCCTGTTCTTGG
AAACGCTGGGCAATGGCAAGCGTACCGTCCGTCGAGCCGTCATCGACAATCAAAATATCC
AAGTTGCGCCAAGTTTGATTCACGACGGCGGCTAATGATTGGGCGAAATATTTTTCTACG
TTGTAGGCGCAAATCAATACGCTGACTAAAGGCTGCAATTTATTCTCCCGATAGGCACGA
TGCCGTCTGAAGGCTTCAGACGGCATATGtatatctccttcttgaaTTCTAACAATTGAT
TGAATGTATGCAAATAAATGCATACACCATAGGTGTGGTTTAATTTGATGCCCTTTTTCA
GGGCTGGAATGTGTAAGAGCGGGGTTATTTATGCTGTTGTTTTTTTGTTACTCGGGAAGG
GCTTTACCTCTTCCGCATAAACGCTTCCATCAGCGTTTATAGTTAAAAAAATCTTTCGGA
ACTGGTTTTGCGCTTACCCCAACCAACAGGGGATTTGCTGCTTTCCATTGAGCCTGTTTC
TCTGCGCGACGTTCGCGGCGGCGTGTTTGTGCATCCATCTGGATTCTCCTGTCAGTTAGC
TTTGGTGGTGTGTGGCAGTTGTAGTCCTGAACGAAAACCCCCCGCGATTGGCACATTGGC
AGCTAATCCGGAATCGCACTTACGGCCAATGCTTCGTTTCGTATCACACACCCCAAAGCC
TTCTGCTTTGAATGCTGCCCTTCTTCAGGGCTTAATTTTTAAGAGCGTCACCTTCATGGT
GGTCAGTGCGTCCTGCTGATGTGCTCAGTATCACCGCCAGTGGTATTTATGTCAACACCG
CCAGAGATAATTTATCACCGCAGATGGTTATCTGTATGTTTTTTATATGAATTTATTTTT
TGCAGGGGGGCATTGTTTGGTAGGTGAGAGATCAATTCTGCATTAATGAATCGGCCAACG
CGCGGGGAGAGGCGGTTTGCGTATTGGGCGCTCTTCCGCTTCCTCGCTCACTGACTCGCT
GCGCTCGGTCGTTCGGCTGCGGCGAGCGGTATCAGCTCACTCAAAGGCGGTAATACGGTT
ATCCACAGAATCAGGGGATAACGCAGGAAAGAACATGTGAGCAAAAGGCCAGCAAAAGGC
CAGGAACCGTAAAAAGGCCGCGTTGCTGGCGTTTTTCCATAGGCTCCGCCCCCCTGACGA
GCATCACAAAAATCGACGCTCAAGTCAGAGGTGGCGAAACCCGACAGGACTATAAAGATA
CCAGGCGTTTCCCCCTGGAAGCTCCCTCGTGCGCTCTCCTGTTCCGACCCTGCCGCTTAC
CGGATACCTGTCCGCCTTTCTCCCTTCGGGAAGCGTGGCGCTTTCTCATAGCTCACGCTG
TAGGTATCTCAGTTCGGTGTAGGTCGTTCGCTCCAAGCTGGGCTGTGTGCACGAACCCCC
CGTTCAGCCCGACCGCTGCGCCTTATCCGGTAACTATCGTCTTGAGTCCAACCCGGTAAG
ACACGACTTATCGCCACTGGCAGCAGCCACTGGTAACAGGATTAGCAGAGCGAGGTATGT
AGGCGGTGCTACAGAGTTCTTGAAGTGGTGGCCTAACTACGGCTACACTAGAAGaACAGT
ATTTGGTATCTGCGCTCTGCTGAAGCCAGTTACCTTCGGAAAAAGAGTTGGTAGCTCTTG
ATCCGGCAAACAAACCACCGCTGGTAGCGGTGGTTTTTTTGTTTGCAAGCAGCAGATTAC
GCGCAGAAAAAAAGGATCTCAAGAAGATCCTTTGATCTTTTCTACGGGGTCTGACGCTCA
GTGGAACGAAAACTCACGTTAAGGGATTTTGGTCATGAGATTATCAAAAAGGATCTTCAC
CTAGATCCTTTTAAATTAAAAATGAAGTTTTAAATCAATCTAAAGTATATATGAGTAAAC
TTGGTCTGACAGTTACCAATGCTTAATCAGTGAGGCACCTATCTCAGCGATCTGTCTATT
TCGTTCATCCATAGTTGCCTGACTCCCCGTCGTGTAGATAACTACGATACGGGAGGGCTT
ACCATCTGGCCCCAGTGCTGCAATGATACCGCGAGACCCACGCTCACCGGCTCCAGATTT
ATCAGCAATAAACCAGCCAGCCGGAAGGGCCGAGCGCAGAAGTGGTCCTGCAACTTTATC
CGCCTCCATCCAGTCTATTAATTGTTGCCGGGAAGCTAGAGTAAGTAGTTCGCCAGTTAA
TAGTTTGCGCAACGTTGTTGCCATTGCTACAGGCATCGTGGTGTCACGCTCGTCGTTTGG
TATGGCTTCATTCAGCTCCGGTTCCCAACGATCAAGGCGAGTTACATGATCCCCCATGTT
GTGCAAAAAAGCGGTTAGCTCCTTCGGTCCTCCGATCGTTGTCAGAAGTAAGTTGGCCGC
AGTGTTATCACTCATGGTTATGGCAGCACTGCATAATTCTCTTACTGTCATGCCATCCGT
AAGATGCTTTTCTGTGACTGGTGAGTACTCAACCAAGTCATTCTGAGAATAGTGTATGCG
GCGACCGAGTTGCTCTTGCCCGGCGTCAATACGGGATAATACCGCGCCACATAGCAGAAC
TTTAAAAGTGCTCATCATTGGAAAACGTTCTTCGGGGCGAAAACTCTCAAGGATCTTACC
GCTGTTGAGATCCAGTTCGATGTAACCCACTCGTGCACCCAACTGATCTTCAGCATCTTT
TACTTTCACCAGCGTTTCTGGGTGAGCAAAAACAGGAAGGCAAAATGCCGCAAAAAAGGG
AATAAGGGCGACACGGAAATGTTGAATACTCATACTCTTCCTTTTTCAATATTATTGAAG
CATTTATCAGGGTTATTGTCTCATGAGCGGATACATATTTGAATGTATTTAGAAAAATAA
ACAAATAGGGGTTCCGCGCACATTTCCCCGAAAAGTGCCACCTGACGTCTAAGAAACCAT
TATTATCATGACATTAACCTATAAAAATAGGCGTATCACGAGGCCCTTTCGTC
Hp2 WP_033756231.1
>Hp2 Helicobacter pylori SA173C WP_033756231.1 LPS biosynthesis protein
[Helicobacter pylori]
SEQ ID NO: 9
MIGVYIISLKESQRRLDTEKLILESNEKFKGRCVFQIFDAISPKHEDFEKFVQELYDAQS
MLKSDWFHSDWCRGELLPQEFGCYLSHYLLWKECVKLNQPVVILEDDVALESNFMQALED
CLKSPFDFVKLFGWYWNFHKTNLRTLPLERDAVESVGETPIEDHVKTEAPETPIENHEVT
PPPNPARDAQQDFIIETQQEELSEPCKIAPQKISFNQVVFKKIKRKLNHFIGNILARTEV
YKKLTGKYDELTGKYDELTGKYDELTGKYDELTGKYESLLAKETNIKETFWERRADSEEE
AFFLEHFYLTSVYVASTAGYYITPKGAKTFIEATERFKIIEPVDMFINNPTYHDVATLTY
LPLPVSLNKHCKISTIQNLKKSDISLSGPKKSYLDNLLYDQLNTRKCLKAFHKYSKQYAP
LKTPKEI
Hc1 WP_104713491.1
> Hcl Helicobacter cetorum GatC WP_104713491.1 lipopolysaccharide
biosynthesis protein [Helicobacter cetorum]
SEQ ID NO: 10
MTQVYIISLKDSKRRLDTEELVSQANIDFEGHCAFHIFDAISPKHKDFEELVREFYEPKS
LLKSDWFHSDCCNGGLLPQELGCFLSHYFLWKKCLELNEPIIILEDDVALEPNFIQALKD
CLKSPFEFVRFCGDYWGYHHTYLNALPIYDNGITPPPPNEESQPIQGSFLAHMVHRVLYF
IIYKIFNRIFHLSLYSIVYRFSRIIKNLQRSHYKKYEKETFFLEHFYLTSVYVGRTAGYY
LTPKGAKAFVDATRNFKMIEPVDMFMDNPAYTDIASITYIPCALSLNEHSLNSTIANQKP
ELLKSYALPKAPKKSYFKNLFYYALNARKRQKAFKKFYEKYAYLKSCKDF
Hf1 WP_023949252.1
>Hf1 Helicobacter_fenneliae_WP_023949252.1 beta-1, 4-galactosyltransferase
[Helicobacter fennelliae]
SEQ ID NO: 11
MFHIFIISLQNSPRRAFMQEQCTHLDRGICQVHFFDAIDERTNAYPALNSKIKPLWNRIY
WGRELSISELGCFGSHYSLWEKCIELNAPIIVLEDDVKLESFFMQGLQEIDQSGFEYVRL
MGLFDVKIEPIKTKSAESKLAESTTKTQHFFKTTDQIAGTQGYYLTPNAAKKFIAKLHSF
CMPVDDYMDCFFIHKVGNILYKPYLIAPAELESTISGRIKQPFSVFKITRECFRLWRKLR
RLLHCL
Cj1 OEV48919.1
>Cj1 Campylobacter_jejuni_OEV48919.1 lipooligosaccharide biosynthesis
glycosyltransferase [Campylobacter jejuni]
SEQ ID NO: 12
MKVFIINLERSLDRKKHMQKQIQKLFEKNPSLKNKLEFIFFKAIDAKNKEHLEFKDHFSW
WGSWILGRELSDGEKACFASHYKLWQECVKLDEPIIILEDDVEFSDEFLNNGIEYIDELL
KSKYEYIRLCYLFDKRLYFLSEGGYYLSFEKLAGTQGYVLQVSAAKKFLKCAKNWIYAVD
DYMDMFYKHNVLNIVKRPLFLKQANFSSVIVEYGRKFSIKLILYKKIAREIFRFYSNILR
LLSIVYIKNRLKLK
Vc1 WP_002023705.1
>Vc1 Vibrio_cholerae_WP_002023705.1 glycosyl transferase [Vibrio cholerae]
SEQ ID NO: 13
MKIYVISLKNSLDRRASIEQQMTSHGLKFEFFDAIDGRIDPPHPLFANYDYIKRLWLTSG
KMPMRGELGCYASHYLLWQKCVELNAPIVVLEDDVIINENFSQYLSIIKDKTNEYGFLRL
EPEVGKCSLFSKESKENYSIAFMDNNWGGTRAYSISPDSARKLILGSQKWSMAVDNYIGC
TYIHKMPSYIFSPSMVEHGVEFETTFQNEKRIRVPLYRKPTREIYSVYKKIRIMMFANEY
KK
Gal WP_018346553.1
>Gal Gallibacterium_anatis_WP_018346553.1 hypothetical protein
[Gallibacterium anatis]
SEQ ID NO: 14
MLPIYVIHIDSATERADSIRQQFDNLKIEFEFFPAINAKKTPNHPLFSHYNAKKHFQRKG
RNLSSGELGCYASHYSTWKKCLELNQPIIVLEDDVTILENFKDIYTNAERIIQKYDFVWL
HKNHRSDDKVIVESIDAFSIAKFYRDYFCAQGYLITPKAAKQLLTYCEEWIYPVDDQMGR
FYENKIENYAIYPACIDHIASMESLIGDDRRGKKKLSFTSKIRREYFNLKDHCRRAWYNF
CFKLGAEVD
03-270 JQ002580 ON_translation
>03-270_JQ002580_ON_translation
SEQ ID NO: 15
MVECQRIPYLGVHLIQVYIISLKESQRRLDTEKLVLESNEKFKGRCVFQIFDAISPKHQD
FEKFVQELYDAQSMLKSDWFHSDYCYQELLPQELGCYLSHYLLWKECVKTDQPIVILEDD
VALESNFMQALEDCLKSPFDFVRLYGHYWGGHKTNLCALPIYTEAEETDYIETEAPIENH
EVTPPPPNPAQDTQQDLINETQQKEPSEPCKIAPPKISFNQVVFKKIKRKLNHFIGNILA
RTEVHKKLVAKYDELTGKYDELTGKYDELTGKYDELTGKYDELTGKYDELTGKYDELTGK
YESLLAKESNIKETFWERRADSEKEAFFLEHFYLTSVYVSTTAGYYLTPKGAKTFIEATE
RFKIIEPVDMFINNPTYHDIANFTYVPCPVSLNKHAFNSTIQNAKKPDISLKPPKKSYED
NLFYNQLNTRKCLRAFHKYSKQYAPLKTPKEV
US6974687_1
>US6974687_1 Sequence 1 from Patent US 6974687 inClaims
gi: 91123855
SEQ ID NO: 16
MISVYIISLKESQRRLDTEKLVLESNEKFKGRCVFQIFDAISPKHEDFEKLLQELYDSSN
LLKSDWFHSDYCYQELLPQEFGCYLSHYLLWKECVKTNQPVVILEDDIALESNFMQALED
CLKSPFDFVRLYGHYWGGHKTNLCALPIYTENENEEVEVPMENHAETEASMEKTPIENHE
VTPPPPNPTQDAQQDCIIETQQDPKELSEPCKIAPQKTSFNPVVFRKIKRKLNRFIGNIL
ARTEVYKNLVSKYDELTGKYDELTGKYDELTGKYDELTGKYDELTGKYDELTGKYDELTG
KYDELTGKYDELTGKYDELTGKYDELTGKYESLLAKEVNIKETFWESRADSEKEALFLEH
FYLTSVYVATTAGYYLTPKGAKTFIEATERFKIIEPVDMFINNPTYHDVANFTYLPCPVS
LNKHAFNSTIQNAKKPDISLKPPKKSYFDNLFYHKFNAQKCLKAFHKYSKQYAPLKTPKE
V
H. pylori GatB WP_075667830.1
>Hp3 Helicobacter pylori_GatB_WP_075667830.1 glycosyltransferase family 25
protein [Helicobacter pylori].
SEQ ID NO: 17
MIQVYIISLKESQRRLDTEKLVLESNEKFKGRCVFQIFDAISPKHQDFEKFVQELYDAQS
MLKSDWFHSDYCYQELLPREFGCYLSHYLLWKECVKTNQPVVILEDDVALESNFMQALED
CLKSPFDFVRLYGHYWGGHKTNLCALPIYTEIEETDYTEIEEAEAPIENHEVPPPPPNST
QDTQQDLINETQQNPKEPSNPCKIAPQKVSFNQVVFKKIKRKLNHFIGNILARTEVYKKL
VAKYDDLTGKYDELTGKYDELTGKYDELTGKYDELTGKYDELTGKYDELTGKYESLLAKE
ANIKETFWERRADSEKEAFFLEHFYLTSVYVSTTAGYYITPKGAKTFIEATERFKIIEPV
DMFINNPTYHDIANFTYVPCPISLNKHAFNSTIQNAKKPDISLKPPKKSYWDNLFYNQLN
TKKCLRAFHKYSKQYDHLKTPKEV
H. cetorum GatD WP_014659558.1
>Hc2 Helicobacter cetorum_GatD_WP_014659558.1 LPS biosynthesis protein
[Helicobacter cetorum].
SEQ ID NO: 18
MISVYIISLKDSKRRLDTEKLVLESNEKFRGHCVFHIFDAISPKHEDFEKLVKELYDASS
LLQSDWFCSSVGNGLSLPELGCYLSHYFLWEECAKLNQPVIVLEDDVALESNFIQALEDC
LKSPFDFVRLYGDYWYFHSTDENTLFTQTANTEKNFKYYIKSRLKNLFKSIPLSQIIIRI
PTKTAELFQRKYFSKREKEALFLEHFYLTSVYVATTAGYYLTPKGAKTFIDATKKFKIIE
PVDMFMDNPTYHDVASLTYVPCALSINGHSENSTIQSQHQGNKKENKKRYKIVLPTPPRK
AYLKRLESYATNAKKRLKAFQQFYEKYAHLESHT

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

1. A composition for use in the production of an oligosaccharide, the composition comprising a bacterium expressing at least one β-1,3-galactosyltransferase enzyme, wherein the amino acid sequence of said at least one enzyme comprises at least 80% identity and up to 100% identity to full length amino acid sequence of SEQ ID NO: 17, 1, 10, or 18.

2. The composition of claim 1, wherein the said at least one enzyme is at least 85% identity and up to 100% identity to full length amino acid sequence of SEQ ID NO: 17.

3. The composition of claim 1, wherein the said at least one enzyme is at least 90% identity and up to 100% identity to full length amino acid sequence of SEQ ID NO: 17.

4. The composition of claim 1, wherein the said at least one enzyme is at least 95% identity and up to 100% identity to full length amino acid sequence of SEQ ID NO: 17.

5. The composition of claim 1, wherein the said at least one enzyme is 100% identical to full length amino acid sequence of SEQ ID NO: 17.

6. A method for producing an oligosaccharide in a bacterium comprising expressing an enzyme in a host bacterium, wherein the amino acid sequence of said enzyme comprises at least 80% identity to GatB (SEQ ID NO:17), thereby producing an oligosaccharide comprising a Gal(β1-3)GlcNAc motif.

7. The method of claim 6, wherein the said enzyme comprises at least 85% identity to GatB (SEQ ID NO:17).

8. The method of claim 6, wherein the said enzyme comprises at least 90% identity to GatB (SEQ ID NO:17).

9. The method of claim 6, wherein the said enzyme comprises at least 95% identity to GatB (SEQ ID NO:17).

10. The method of claim 6, wherein the said enzyme is 100% identical to GatB (SEQ ID NO:17).
